(19) United States
(12) Patent Application Publication
(10) Pub. No.: US 2014/0316519 A1
(43) Pub. Date: Oct. 23, 2014

(54) CARDIAC VALVE WITH SHIELDS FOR TISSUE RETRACTION

(71) Applicant: VALVEXCHANGE, INC., GREENWOOD VILLAGE, CO (US)

(72) Inventors: Ivan Veseley, Larkspur, CO (US); Todd D. Campbell, Petaluma, CA (US); Christopher Michael Sprague, Broomfield, CO (US); Dean Carpenter, Lafayette, CO (US)

(73) Assignee: VALVEXCHANGE, INC., GREENWOOD VILLAGE, CO (US)

(21) Appl. No.: 14/356,323

(22) PCT Filed: Nov. 5, 2012
(86) PCT No.: PCT/US2012/063606

§ 371 Date: May 5, 2014

Related U.S. Application Data
(60) Provisional application No. 61/556,120, filed on Nov. 4, 2011.

(51) Int. Cl. A61F 2/24

(52) U.S. Cl. CPC A61F 2/2427
USPC 623/2.11

(57) ABSTRACT

A tissue retraction system for a bioprosthetic cardiac valve base configured for attachment to a separate leaflet set includes a plurality of retraction shields releasably attached to the valve base. The retraction shields are configured to restrain tissue within a surgical site. The retraction shields may be flexible and transparent. Shield extenders can be attached to the retraction shields and used to further secure the retraction shields to a device for providing tissue retraction during a surgical procedure.

### CROSS REFERENCE TO RELATED APPLICATION

This application claims the benefit of priority under 35 U.S.C. §119(e) to U.S. Application No. 61/556,120, filed 4 Nov. 2011, titled “Cardiac valve with shields for tissue retraction,” and which is hereby incorporated by reference in its entirety.

### TECHNICAL FIELD

The technology described herein relates to bioprosthetic cardiac valve systems and surgical tools for implanting the same.

### BACKGROUND

During the insertion of most surgically implantable cardiac valves, the diameter of the sewing cuff is typically greater than the diameter of the sinotubular junction of the ascending aorta. With conventional valves, the surgeon typically pushes and twists the valve into place until it is forced through this somewhat narrow space. Occasionally, external retractors may be used to spread apart the tissue to get the valve to seat into the aortic root.

With a two part valve, i.e., a valve with a permanent base and a replaceable leaflet system such as described, for example, in U.S. Pat. No. 6,569,196, the base can be forced into place like other valves, but the leaflets are somewhat more delicate and need to be protected from external compression. Because of the tight space within patients' chests, external retractors are not convenient, particularly as surgical incisions get smaller and surgeons use more minimally invasive approaches.

The information included in this Background section of the specification, including any references cited herein and any description or discussion thereof, is included for technical reference purposes only and is not to be regarded as subject matter by which the scope of the invention as defined in the claims is to be bound.

### SUMMARY

The technology disclosed herein includes a system of strips of a biocompatible material that are secured to a valve base of a two-part bioprosthetic valve and can be used in various ways to make the valve implant procedure more convenient. The system may include one or a combination of several functional components. Three or more shields may be sewn onto the valve base during manufacture and can be removed after implantation of the valve base by simply cutting each of the sutures and lifting the shields out. Shield extenders may be secured to the shields and also via sutures to a system for managing sutures, such as a Gabbay-Frater suture guide. Traction can be applied to the sutures to provide tissue retraction from the shields and shield extenders. The tissue retraction shields attached to the valve base may aid in minimizing the invasiveness of the procedure by reducing the size of the surgical incision to 7 cm or less because the available tissue retraction provided by the shields greatly opens up the sight into the surgical field.

In one exemplary embodiment of the system disclosed herein, the shields are secured to a base holder by an annular tab clip. The tab clip may be a simple ring that slips over the shaft of the base holder, which is used for insertion of the valve base. Each of the shields may have retention tabs that can fit into slots on the tab clip. Each shield extender may hook under a suture than runs through the corresponding shield.

In another exemplary embodiment of the system disclosed herein, the shields are secured to a base holder by a shield cap. The shield cap may slip over the handle, which is used for insertion of the valve base. Each of the shields may have retention tabs that are secured between locking slots of the shield cap and locking extensions of the base holder.

In still another exemplary embodiment of the system disclosed herein, the shields are secured to a valve base via a locking region that can fit into a locking trough on the base holder. Each of the shields may fit over a leg of a base holder.

In one implementation, a prosthetic heart valve implantation system includes a valve base configured for in vivo implantation and to hold a valve leaflet set. The system further includes a tissue retraction shield removably attached to the valve base.

In another implementation, a bioprosthetic cardiac valve base is configured for attachment to a separate leaflet set. The valve base may have a substantially annular valve seat provided with a biocompatible fabric coating. The valve base may also have a plurality of hooks extending proximally from the valve seat and configured for attachment with a valve leaflet set. A plurality of retraction shields for restraining tissue within a surgical site may be realeasably secured to the valve seat. Each retraction shield may be positioned adjacent to and extend over a respective one of the plurality of hooks.

In still another implementation, a tissue retraction system is disclosed for use in conjunction with a bioprosthetic cardiac valve. The tissue retraction system may include an elongated handle, a valve base holder, a valve base, a plurality of shields, and a shield retainer. The valve base holder may be attached to a distal end of the handle. The valve base may be realeasably secured to the valve base holder. The valve base may include a substantially annular valve seat provided with a biocompatible fabric coating and a plurality of hooks configured for connection with a valve leaflet set. A plurality of shields are provided for restraining tissue within a surgical site. Each shield may be positioned adjacent to and extending over a respective one of the plurality of hooks. A shield retainer may be moveably positioned on the handle above the valve base holder. A base end of each of the shields may be releasably secured to the valve seat. A top end of each of the shields may be releasably secured to the shield retainer.

In a further implementation, a tissue retraction system is disclosed for use in conjunction with a bioprosthetic cardiac valve. The tissue retraction system may include an elongated handle, a valve base holder, a valve base, and a plurality of retraction shields. The valve base holder may be attached to a distal end of the handle. The valve base may be realeasably secured to the valve base holder. The valve base may include a substantially annular valve seat provided with a biocompatible fabric coating and a plurality of hooks configured for connection with a valve leaflet set. The valve base may include a plurality of legs, which may be positioned adjacent to and extending over a respective one of the plurality of hooks. A plurality of retraction shields are provided for restraining tissue within a surgical site. Each retraction shield may be positioned adjacent to and extending around a respective one of the plurality of legs. A base end of each of the retraction shields may be releasably secured to the valve seat. A top end of each of the retraction shields may be releasably secured to the base holder.

In another implementation, a method of implanting a prosthetic cardic valve is provided. The method includes suturing a valve base to an implantation site on a heart and retracting tissue surrounding the implantation site by placing traction on one or more retraction shields removably attached to the valve base.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. A more extensive presentation of features, details, utilities, and advantages of the present invention as defined in the claims is provided in the following written description of various embodiments of the invention and illustrated in the accompanying drawings.

### BRIEF DESCRIPTION OF THE DRAWINGS

FIG. 1 is a front elevation view of an aortic root with a bioprosthetic cardiac valve inserted therein.

FIG. 2 is a top isometric view of a two-part, bioprosthetic cardiac valve.

FIG. 3A is a front elevation view of a valve introducer system according to one embodiment in which shields are attached to a valve base and operably connected to a base holder via a tab clip.

FIG. 3B is front isometric view of the valve introducer system of FIG. 3A.

FIG. 4 is a front isometric view of the valve base and shields of FIGS. 3A and 3B separated from the base holder and base holder shaft.

FIG. 5A is a top plan view of a shield extender.

FIG. 5B is a front isometric view of the valve base and shields of FIGS. 3A and 3B separated from the base holder and base holder shaft, with the shield extender of FIG. 5A attached to shields.

FIG. 6 is a schematic diagram of an intra-operative procedure in which the shield extenders of FIG. 5A are attached to a Gabbay-Frater suture guide.

FIG. 7 is a front elevation view of a valve introducer system according to another embodiment in which shields are secured to a valve base and to a base holder via a shield cap.

FIG. 8 is a partial front isometric view of the valve introducer system of FIG. 7.

FIG. 9A is a right elevation view of the valve introducer system of FIG. 7.

FIG. 9B is a left elevation view of the valve introducer system of FIG. 7.

FIG. 10 is a rear elevation view of the valve introducer system of FIG. 7.

FIG. 11 is an exploded view of the valve introducer system of FIG. 7.

FIG. 12 is front left isometric view of a shield of the valve introducer system of FIG. 7.

FIG. 13A is left rear elevation view of a base holder of the valve introducer system of FIG. 7.

FIG. 13B is top front isometric view of the base holder of FIG. 13A.

FIG. 14A is front right elevation view of a shield cap of the valve introducer system of FIG. 7.

FIG. 14B is front left isometric view of the shield cap of FIG. 14A.

FIG. 14C is bottom left isometric view of the shield cap of FIG. 14A.

FIG. 14D is bottom rear isometric view of the shield cap of FIG. 14A.

FIG. 15 is an isometric view in cross section taken along line 15-15 in FIG. 7.

FIG. 16A is a front isometric view of the valve introducer system of FIG. 7 in which sutures secure the shields to the valve base.

FIG. 16B is a front isometric view of the valve introducer system of FIG. 16A with the shields released from the shield cap and base holder.

FIG. 17 is a top isometric view of the valve introducer system of FIG. 7 with the handle removed.

FIG. 18 is a front elevation view of a valve introducer system according to another embodiment in which shields are secured over legs of a base holder to a valve base.

FIG. 19 is a front isometric view of the valve introducer system of FIG. 18 with the handle removed.

FIG. 20A is a front elevation view of the valve introducer system of FIG. 19.

FIG. 20B is a right elevation view of the valve introducer system of FIG. 19.

FIG. 20C is a bottom plan view of the valve introducer system of FIG. 19.

FIG. 21 is a front isometric view of the valve base of the valve introducer system of FIG. 19.

FIG. 22 is front left isometric view of a shield of the valve introducer system of FIG. 19.

FIG. 23A is a top front isometric view of a base holder of the valve introducer system of FIG. 19.

FIG. 23B is a bottom isometric view of the base holder of FIG. 23A.

### DETAILED DESCRIPTION

A tissue retraction system for use in conjunction with the implantation of a prosthetic cardiac valve is disclosed herein. The tissue retraction system may be generally understood as strips of a biocompatible material that function as shields and are temporarily secured to a valve base of a two-part bioprosthetic valve during implantation. The shields may be used in various ways to make the valve implantation procedure more convenient. When the valve base is placed in situ, the shields may cover hooks of the valve base designed to connect with a separate removable leaflet set.

The shields may be relatively short for ease of packaging. In order for them to be effective soft tissue retractors, however, they may need to be extended. To accomplish this, a shield extender may be secured to a shield to increase the effective length of the shield. Once the shield extenders are secured to the shields, they can also be attached to a conventional Gabbay-Frater suture guide via sutures. Traction can be applied to the sutures that secure the shield extenders. This traction can be adjusted to provide the proper amount of exposure of the valve base during suturing by way of soft tissue retraction, and also to effectively lift the annulus towards a more vertical orientation for the installation of the leaflet set.

In one embodiment, the shields may be secured to the shaft of a base holder for implantation by way of a tab clip, into which retention tabs extending from the shields are inserted. In another embodiment, retention tabs extending from the shields may be secured to a base holder by a shield cap. In still another embodiment, a locking region of the shields may be secured to a base holder by a locking trough.

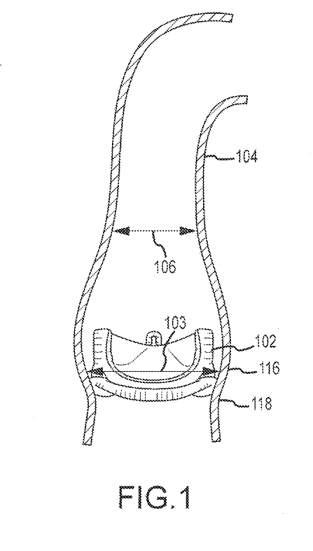

FIG. 1 depicts an ascending aorta 104 with a bioprosthetic cardiac valve 102 inserted therein in replacement of a natural aortic valve. The diameter of the ascending aorta 104 above the bioprosthetic cardiac valve 102 at the sinotubular junction 106 is typically of smaller diameter than the diameter 103 of the bioprosthetic cardiac valve 102 itself.

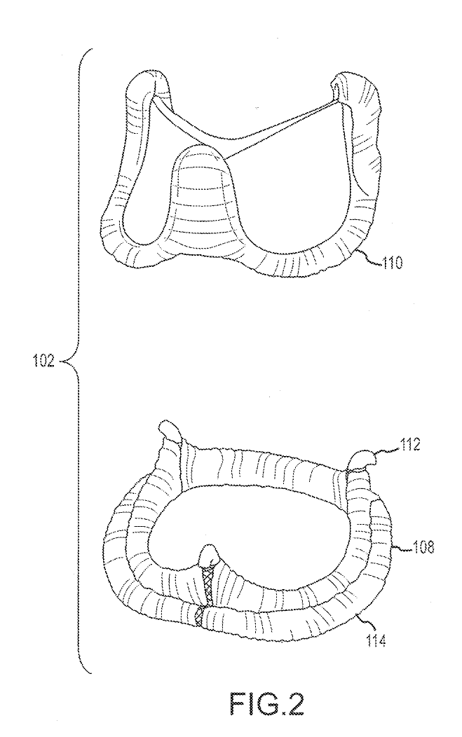

FIG. 2 illustrates a two-part bioprosthetic cardiac valve 102 comprised of a permanent base 108 and a removable leaflet set 110. The valve base 108 may be inserted into and attached to the aortic root 116 at the outlet of the left ventricle (see FIG. 1). The leaflet set 110 may be secured to corresponding hooks 112 extending from the valve base 108 after the valve base 108 has been sutured in place. This two-part configuration of the cardiac valve 102 has multiple advantages, including, for example, allowing for faster implantation of the valve base 108 by the surgeon because the leaflet set 110 is not in the way, improving the ability to check for gaps between the annulus 118 (see FIG. 1) of the aortic root 116 and the sewing cuff 114 of the valve base 108 and fixing them from underneath, and permitting exchangeability of the leaflet set 110 for a new leaflet set, such as when the leaflet set 110 becomes worn out.

The valve base 108, including the sewing cuff 114, may be substantially circular to fit in the annulus 118. The valve base 108 may have smooth, curved edges. The underside of the valve base 108 may create a flat plane or it may undulate such that it falls above and/or below a plane, and so that it conforms to the shape of the aortic root 116. The top side of the valve base 108 rises in three places to form or secure a corresponding number of hooks 112. The hooks 112 are substantially evenly spaced around the circumference of the valve base 108. The hooks 112 face away from the interior of the space defined by the valve base 108 and may secure a leaflet set 110.

The valve base 108 is comprised of one or more biocompatible materials. The biocompatible materials may be any biocompatible materials known in the art. By way of example but not limitation, the core of the valve base 108 may be constructed of polyaryletherketone (PEEK) or medical grade stainless steel and it may be partially or completely covered with a woven polyester fabric. The fabric form of the biocompatible material covering the sewing cuff 114 allows for the passage of a needle and suture thread.

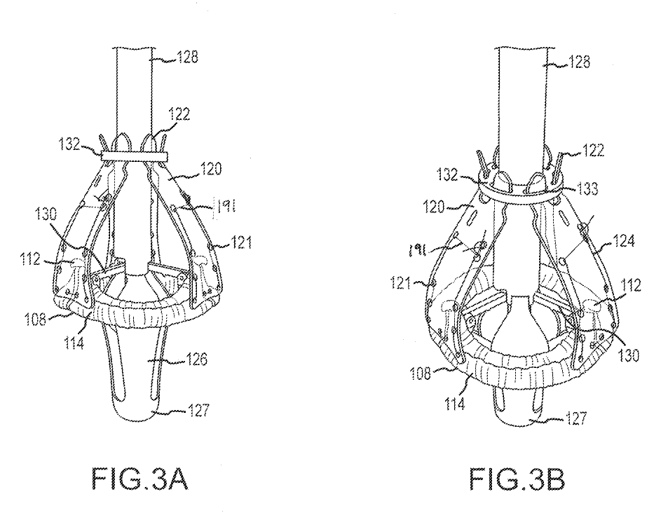

Referring now to FIG. 3A and FIG. 3B, an exemplary valve base 108 is secured to a base holder 126. The base holder 126 may have retractable arms 130 that secure the base holder 126 to the valve base 108 via pins (not shown) that fit into pin receiving holes 382c (see FIG. 21) in the valve base 108. The base holder 126 may be comprised of more than one assembled pieces, including, but not limited to, a shaft 128 and retractable arms 130. The terminal end 127 of the base holder 126 may extend beyond the plane of the underside of the valve base 108, may be flush with the plane of the underside of the valve base 108, or may terminate above the plane of the underside of the valve base 108. In one exemplary embodiment, the terminal end may function as a placement guide to center the valve base 108 within the opening of the aortic root 118. The retractable arms 130 may retract into or alongside the base holder 126 once the valve base 108 is at least partially attached in place within the aortic root 118. There may be the same number of retractable arms 130 as hooks 112.

One or more, for example three, retraction shields 120 may be attached to the valve base 108. The retraction shields 120 may be formed of any sturdy and flexible biocompatible material known in the art. The material may be one of any of numerous plastics including, for example polycarbonate, polyetherimide, polypropylene, polyphenylsulfone, polytetrafluoroethylene (PTFE) or PEEK. The material may be transparent, translucent, or opaque. The retraction shields 120 may have smooth surfaces and smooth and/or rounded edges. The retraction shields 120 may be substantially rectangular in shape with a lateral width at least wide enough to cover the width of a hook 112. Any number of holes 121 may be preformed in the retraction shields 120. By way of example but not limitation, eleven holes 121 may be preformed in the retraction shields 120 towards the outer edges of the retraction shields 120. The preformed holes 121 allow for passage of a needle and thread, such as suture thread. Sutures 124 may be inserted through some or all of the preformed holes 121. One or more sutures 124 may secure the bottom end of the retraction shields 120 to the valve base 108, for example, to the sewing cuff 114.

The bottom ends of the retraction shields 120 may be positioned anywhere along the valve base 108, for example in the Nadir portions of the valve base 108. In another example, the retraction shields 120 may be positioned radially external to and extend over and above the hooks 112 of the valve base 108. The connection between the bottom ends of the retraction shields 120 and the valve base 108 may function as a hinge to permit the retraction shields 120 to be pushed or bent inward towards the base holder shaft 128 and/or pushed or bent outward against tissue during implantation of the valve base 108, which may restrain tissue at or near the implantation site.

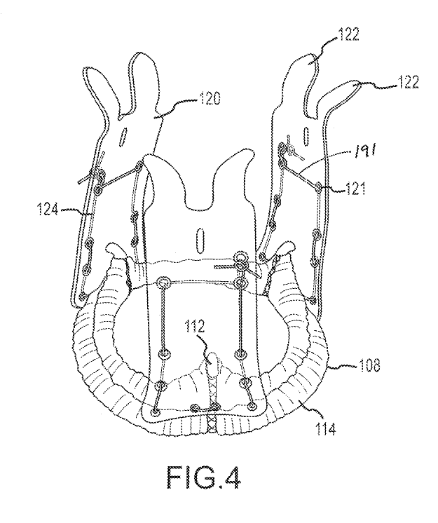

A top end of each shield 120 may terminate in one or more retention tabs 122. In one exemplary embodiment as shown in FIG. 4, the top end of each shield 120 may bifurcate into two retention tabs 122. The retention tabs 122 may be any shape and size. The retention tabs 122 may be used to secure the retraction shields 120 against the shaft 128 of a base holder 126 during implantation of the valve base 108.

The retention tabs 122 may be secured by way of an annular tab clip 132 that encircles the base holder shaft 128. The tab clip 132 may be formed of any biocompatible material known in the art including, but not limited to, plastic. The annular configuration of the tab clip 132 and its position around the shaft 128 above the base holder 126 ensures that the tab clip 132 cannot fall down into the patient. The tab clip 132 may be formed with a number of slots 133 that receive the retention tabs 122. The retention tabs 122 may extend through the slots 133. The number of slots 133 may equal the number of retention tabs 122. The retention tabs 122 may be pre-bent or may be bendable. When the retention tabs 122 are positioned through the slots 133 of the tab clip 132, they may be bent inwards toward or outwards from the shaft 128 to a greater or lesser degree to adjust the degree of security between the retraction shields 120 and base holder 126.

As noted, the bottom end of the retraction shields 120 may be secured to the valve base 108 by sutures 124. Securing both the top and bottom ends of the retraction shields 120 permits them to be pulled tight against the shaft 128 and also effectively covers the hooks 112 on the valve base 108. The presence of the retraction shields 120 during suturing of the valve base 108 allows the knots to quickly slide over the retraction shields 120 as the knots are pushed down against the sewing cuff of the valve base 108. The presence of the retraction shields 120 prevents sutures from getting caught on the hooks 112 during the tying of the knots during the implantation of the valve base 108. Once all the knots are tied, the retraction shields 120 may be released from the shaft 128 by pulling the tab clip 132 upwards until the retention tabs 122 pull out of slots 133 in the tab clip 132.

After the top ends of the retraction shields 120 are released from the shaft 128 and the valve base 108 is released from the base holder 126, the base holder 126 including the tab clip 132 can be removed from the surgical field, as illustrated in FIG. 4. The bottom end of the retraction shields 120 are still secured to the valve base 108, for example by sutures 124. The retraction shields 120 are free to be pushed or bent outward against tissue during further implantation of the valve base 108 and connection of the valve leaflet set 110. Once the valve 102 is assembled in the patient, the sutures 124 can be cut to release the retraction shields 120 to permit their removal from the surgical site.

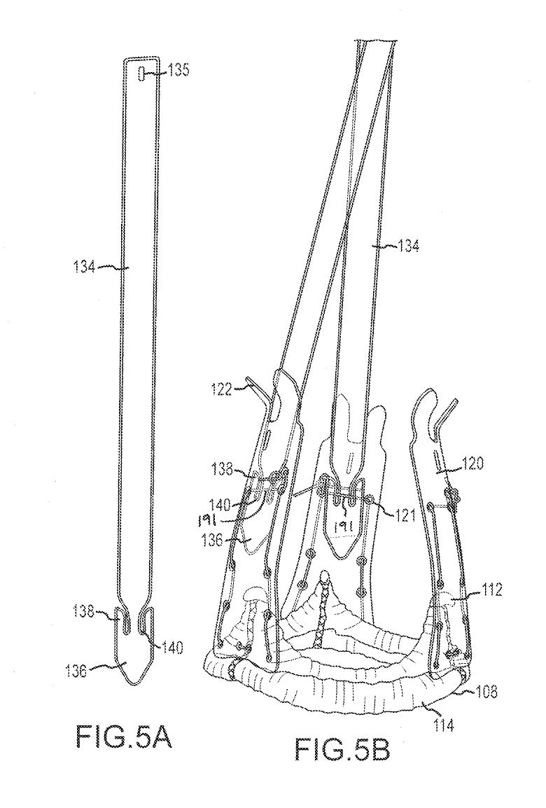

The effective length of a retraction shield 120 may be extended by a shield extender 134, as shown in FIG. 5A. The number of shield extenders 134 may equal the number of shields 120. A shield extender 134 may be formed of any sturdy and flexible biocompatible material known in the art. The material may be one of any of numerous plastics including, for example, polycarbonate, polyetherimide, polypropylene, polyphenylsulfone, polytetrafluoroethylene (PTFE) or PEEK The material may be transparent, translucent, or opaque. The shield extenders 134 may have smooth surfaces and smooth and/or rounded edges.

The shield extenders 134 may be any shape, width, and length. The shield extenders 134 may be substantially rectangular in shape with a length longer than width. The width may be at least wide enough to restrain tissue within the surgical site. A shield extender 134 may terminate in a head 136, wherein the shape of the head 136 helps to secure a shield extender 134 to a retraction shield 120. The head 136 may be substantially arrow-shaped. The arrow-shaped head 136 may be formed with two tail fingerhooks 138 extending rearward from lateral sides of the head 136 to further form gaps 140 between the tail fingerhooks 138 and the main body of the shield extender 134.

As shown in FIG. 5B, the head 136 of the shield extender 134 may be attached to the retraction shield 120 secured to the valve base 108 by sliding the head 136 of the shield extender 134 under the horizontal suture span 191 adjacent the top end of the retraction shield 120. The fingerhooks 138 of the arrow-shaped head 136 capture the horizontal suture span 191 within the gap 140 with the fingerhooks 138 on top and the main body of the shield extender 134 underneath the horizontal suture span 191. The shield extender 134 may be secured to the front or back of the retraction shield 120.

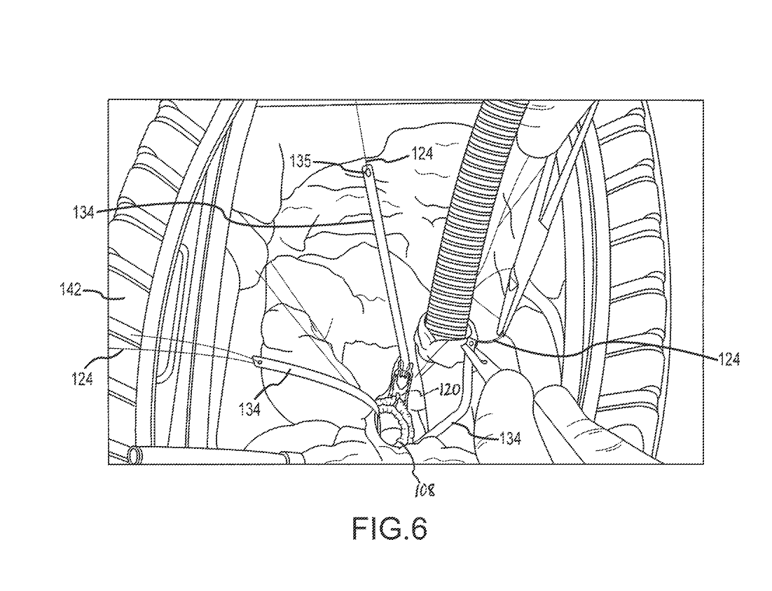

Any number of holes 135 may be preformed in the shield extender 134. By way of example but not limitation, one hole 135 may be preformed in the shield extender 134 toward the tail of the shield extender 134. The preformed hole 135 allows for passage of a needle and thread, such as suture thread. Sutures 124 may be inserted through some or all of the preformed holes 135 (see FIG. 6). While in the exemplary embodiment shown, one hole 135 is preformed in the shield extender 134 toward the tail of the shield extender 134, one or more preformed holes 135 may be positioned elsewhere along the shield extender 134 to maximize the utility and flexibility of the shield extender 134.

FIG. 6 shows an intra-operative procedure wherein a needle and thread are passed through a preformed hole 135 in the tail of a shield extender 134, which has already been secured to a retraction shield 120. Both on the left and distal sides of the surgical field, other shield extenders 134 are shown with one or more sutures 124 already securing each shield extender 134 to a system for holding sutures 142, for example a Gabbay-Frater suture guide. Securing the shield extenders 134 to a system for holding sutures 142 allows traction to be applied to the retraction shields 120 and shield extenders 134 via traction on the sutures 124. Traction on the retraction shields 120 and shield extenders 134 aids in implantation of the valve 102, for example, by providing the proper amount of exposure of the surgical site by way of soft tissue retraction. The traction may also provide benefit by effectively lifting the annulus 118 towards a more vertical orientation for the later installation of a leaflet set 110.

By way of example, but not limitation, the valve introducer system and components of FIGS. 3-5 may be inserted into a patient undergoing aortic valve replacement surgery according to the following procedure. Note that a similar procedure may be followed for the implantation of an artificial pulmonary valve. After the natural aortic valve is resected, the valve base 108, its base holder 126, and the associated shields 120 are positioned above the surgical space buy holding the shaft 128. Sutures are then passed through the annular tissue and the sewing cuff 114 and suture apertures (hidden by the sewing cuff 114) in the valve base 108. Once all the sutures are attached between the annulus 118 and the valve base 108, the valve base 108 is pushed down into the patient's annulus 118 by pushing the base holder 126 with the shaft 128. The sutures are then tied off by way of knots that are pushed down against the surface of the sewing cuff 114. The sutures may then be cut off, or left in place and organized by using the appropriate system for holding sutures 142, such as a Gabbay-Frater suture guide.

Once the valve base 108 is fully secured to the aortic root 116 by sutures through the sewing cuff 114, the retraction shields 120 may then be released from the shaft 128 by pulling the tab clip 132 upward along the shaft 128 of the base holder 126 until the retention tabs 122 of the retraction shields 120 pull out of slots 133 in the tab clip 132. The retractable arms 130 may then be released from the valve base 108 and retracted into or alongside the base holder 126. The base holder 126 may then be removed from the surgical field. It should be noted that the base holder 126 can be removed at any desirable point during the procedure and need not be removed immediately after the retraction shields 120 are released from the shaft 128

The retraction shields 120 may be bent away from the base holder 126. The shield extenders 134 may be attached to the retraction shields 120 by sliding the head 136 of a shield extender 134 under the horizontal suture span 191 adjacent the top end of a retraction shield 120 such that the fingerhooks 138 capture the horizontal suture span 191.

The shield extenders 134 may be attached to a Gabbay-Frater suture guide via a suture 124 through a preformed hole 121 at the tail of a shield extender 134, or by clamping them with surgical clamps. Traction may be applied to the sutures 124 that secure the shield extenders 134. This traction can be adjusted to provide the proper amount of exposure of the surgical site for additional suturing of the sewing cuff 114 of the valve base 108 by way of soft tissue retraction, and also to effectively lift the annulus 118 of the aortic root 116 toward a more vertical orientation for the later installation of a leaflet set 110.

The leaflet set 110 may be secured to hooks 112 extending from the valve base 108. Once the valve 102 is assembled in the patient, the sutures 124 can be cut to release both the retraction shields 120 and the attached shield extenders 134 to permit their removal from the surgical site.

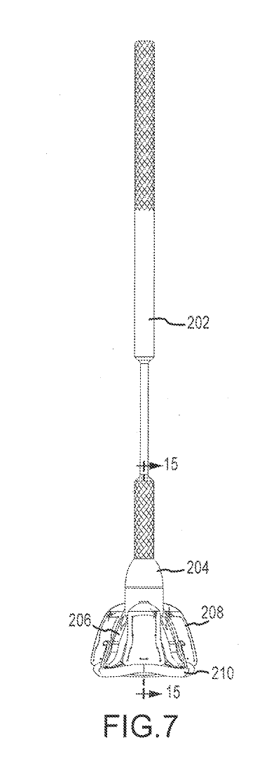

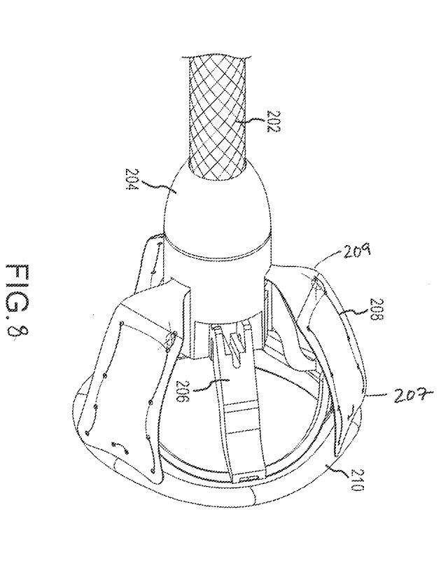

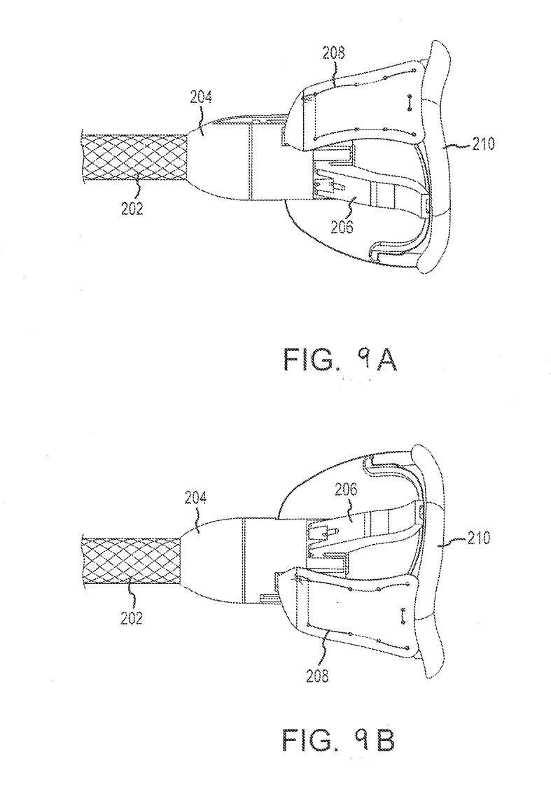

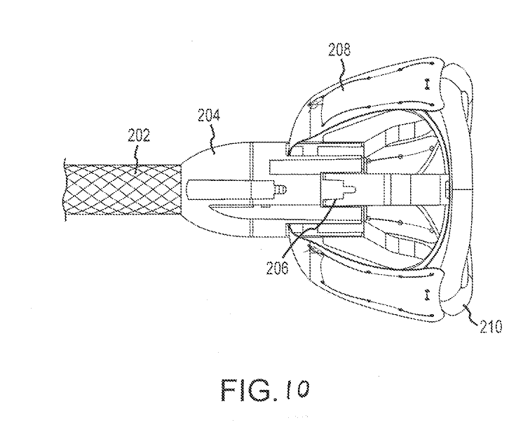

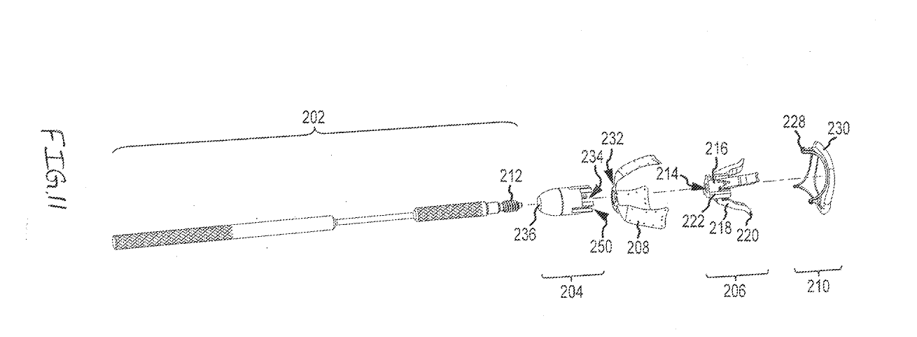

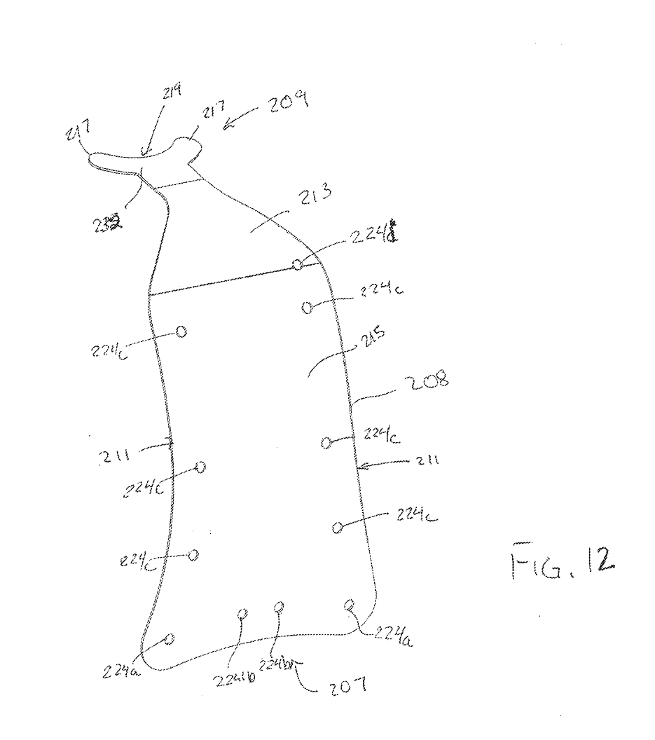

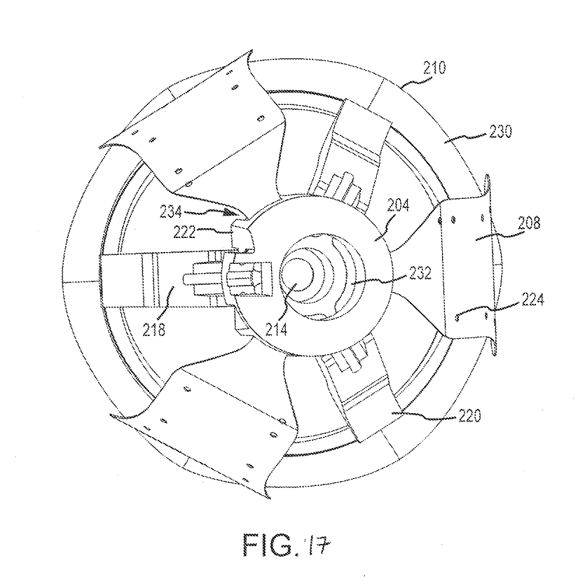

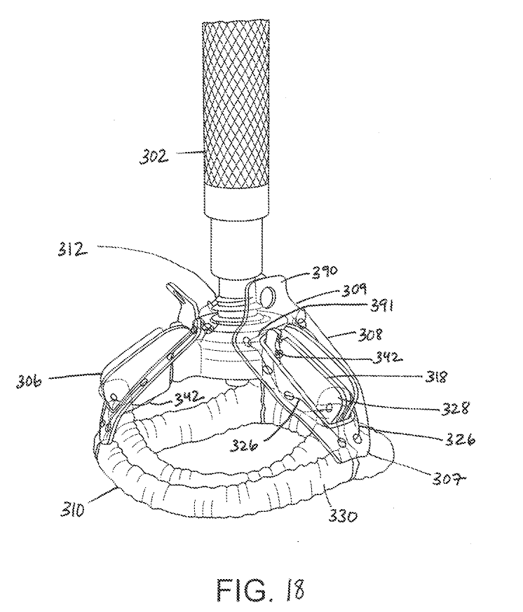

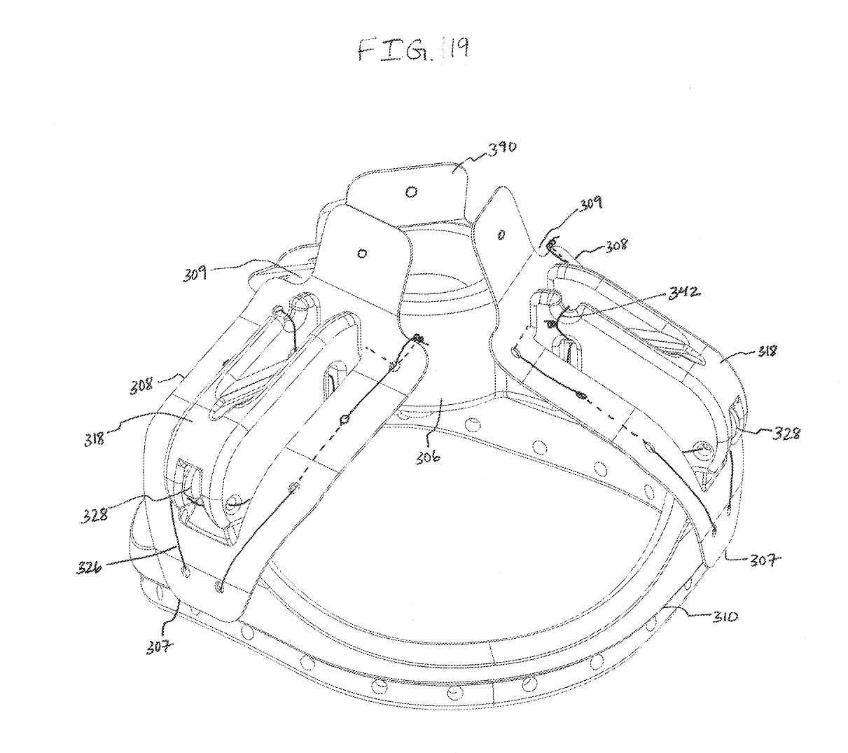

FIGS. 7-17 show a valve introducer system according to another exemplary implementation. With initial reference to FIGS. 7-11, a valve base 210 may be secured to a base holder 206 and the base holder 206 may be secured to an handle 202. One or more retraction shields 208 are secured at their base ends 207 to the valve base 210 and the free top ends 209 of the retraction shields 208 may be retained by a shield cap 204, which fits over or around the handle 202 and interfaces with the base holder 206.

The exemplary valve base 210 is of the same form and construction as the valve base 108 of FIG. 2. As in the prior embodiment, the valve base 210 may be inserted into and attached to the aortic root 116 (see FIG. 1). A leaflet set 110 (see FIG. 2) may be secured to corresponding hooks 228 extending from the valve base 210 after the valve base 210 has been secured.

The valve base 210, including the sewing cuff 230, may be substantially circular to fit in the annulus 118 (see FIG. 1). The valve base 210 may have smooth, curved edges. The underside of the valve base 210 may create a flat plane or it may undulate such that it falls above and/or below a plane, and so that it conforms to the shape of the aortic root 116. The top side of the valve base 210 rises in three places to form or secure a corresponding number of hooks 228. The hooks 228 are substantially evenly spaced around the circumference of the valve base 210. The hooks 228 face away from the interior of the space defined by the valve base 210 and may secure a leaflet set 110 (see FIG. 2).

As shown in FIG. 12, each of the retraction shields 208 may have a substantially rectangular area 215 and a triangular area 213 at the top end 209 of each shield 208. The width of the base end 207 of each shield 208 may be at least wide enough to cover the width of the hook 228 of the valve base 210 and may be so wide that it extends beyond the outer circumference of the valve base 210 (see FIG. 18). The width of the retraction shields 208 may vary over the length of the retraction shields 208. As shown in FIG. 12, the base end 207 of each shield 208 may have a slight concave curve and transition at rounded lateral corners to substantially vertical, but slightly concave, sidewalls 211. The sidewalls 211 of each shield 208 may taper inward toward the top end 209 to form the generally triangle-shaped area 213. The top end 209 of each shield 208 may further be formed as a retention tab 232 that bifurcates into a Y-shape to form two lateral ears 217. The retention tabs 232 may be used to secure the retraction shields 208 against the base holder 206 during implantation of the valve base 210. A top edge 219 of the tab 215 between the ears 217 may be formed as a concave curve with a radius that conforms to a radius of the handle 202 (see FIG. 15). The retraction shields 208 may be formed of any sturdy and flexible biocompatible material known in the art. For example, the material may be plastic. The material may be transparent, translucent, or opaque. The retraction shields 208 may have smooth surfaces and smooth and/or rounded edges.

Any number of holes 224 may be preformed in the retraction shields 208. By way of example, but not limitation, eleven holes 224 may be preformed in the retraction shields 208 towards the outer edges of the retraction shields 208. In the exemplary embodiment shown in FIG. 12, four holes are formed adjacent the base end 207 with two corner holes 224a spread laterally toward the bottom corners and two inner holes 224b adjacent each other symmetric with a vertical center of the retraction shield 208.

Additionally, three sidewall holes 224c may extend along and adjacent each of the sidewalls 211 in the rectangular area 215 of each shield 208 above the corner holes 224a along the base end 207. An additional upper hole 224d may be formed adjacent one of the sidewalls 211 above one set of the sidewall holes 224c in an area of transition between the generally rectangular area 215 of the retraction shield 208 and the triangular area 213 of the retraction shield 208. The preformed holes 224 allow for passage of a needle and suture thread 226. The suture thread 226 may be inserted through some or all of the preformed holes 224.

With reference to FIGS. 7-10 and 16A and 16B, the retraction shields 208 may be attached to the valve base 210. The base ends 207 of the retraction shields 208 may be positioned anywhere along the valve base 210, for example in the Nadir portions of the valve base 210. In another example, as in the figures, the retraction shields 208 may be positioned radially external to and extend over and above the hooks 228 of the valve base 210. One or more sutures 226 may secure the base ends 207 of the retraction shields 208 to the valve base 210, for example, to the sewing cuff 230. As shown in the figures, a single suture thread 226 may be threaded through the holes 224 in each of the retraction shields 208 in a pattern around the perimeter of the retraction shields 208 and tied off adjacent the upper hole 224d adjacent the triangular area 213. In the exemplary embodiment shown, along the base end 207, the suture thread 226 may pass through the corner holes 224a and travel through the fabric cover of the valve base 210, the sewing cuff 230, and/or preformed holes 382 in the valve base 210 (see FIG. 21). The suture thread 226 may pass outward through each of the inner holes 224b so that the suture thread 226 rests on the outer surface of the retraction shields 208. The retraction shields 208 are positioned over the hooks 228 of the valve base 210 with the inner holes 224b positioned on each side of a respective hook 228. Securing both the top end 209 and base end 207 of the retraction shields 208 permits the retraction shields 208 to be pulled against the base holder 206 and also effectively cover the hooks 228 on the valve base 210 during portions of an implantation procedure. Only the base ends 207 of the retraction shields 208 may be connected to the valve base 210 to permit the retraction shields 208 to be pushed or bent inward towards the base holder 206 and shield cap 204 and/or pushed or bent outward against tissue during implantation of the valve base 210, which may restrain tissue within the surgical site.

With reference to FIG. 11, the handle 202 may be formed of any sturdy biocompatible material. In an exemplary embodiment, the handle 202 may be made of stainless steel or titanium. The handle 202 may be constructed such that it is easy and comfortable for the human hand, including a gloved hand, to grip or hold. The handle 202 may be formed as an elongated shaft. The circumference of the handle 202 may vary over the length of the handle 202. The handle 202 may have a smooth surface or may be fully or partially cross-hatched, grooved, or ribbed. The handle 202 may be secured to the base holder 206 by a terminal threaded end 212. The handle 202 may be formed as a single piece or more than one assembled pieces.

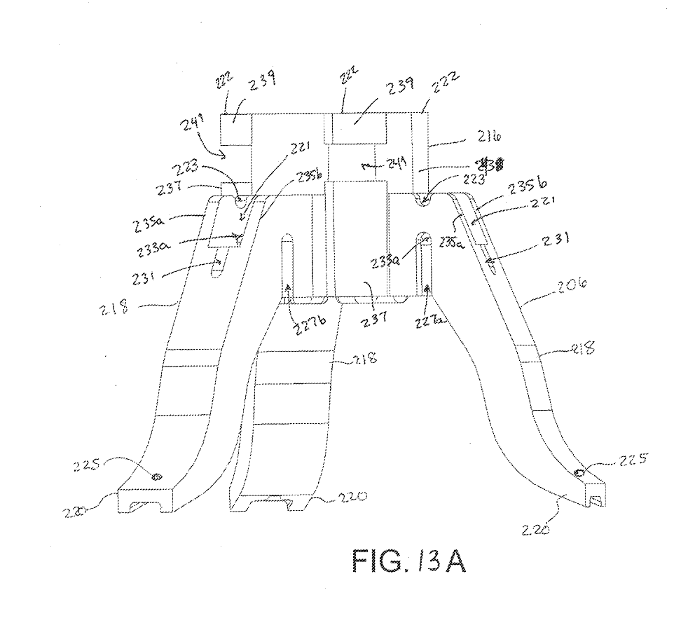

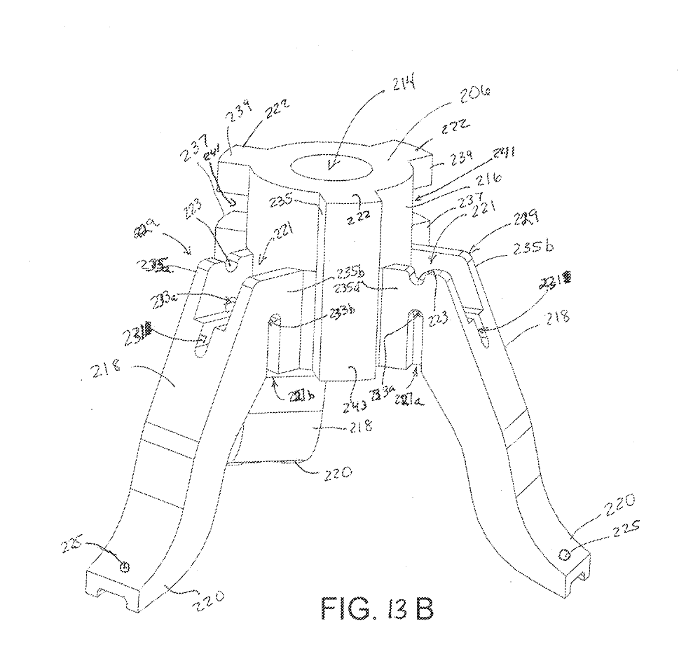

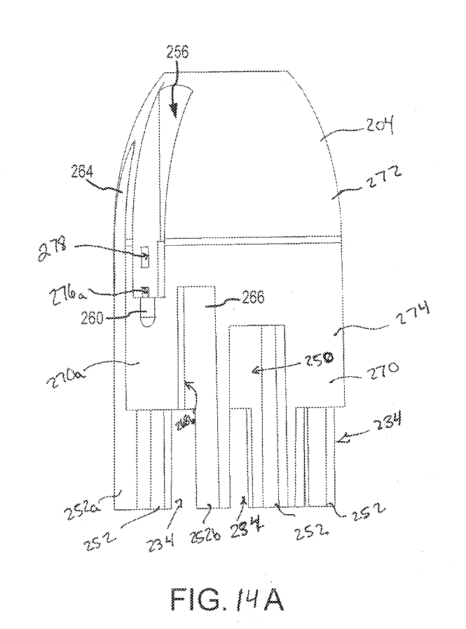

The base holder 206 is depicted in greater detail in FIG. 13A and FIG. 13B and may be formed of any biocompatible material known in the art including, but not limited to, plastic. A plurality of legs 218, for example, three, may extend outward and downward from a generally cylindrical core 216 of the base holder 206. The legs 218 may be substantially evenly spaced around the core 216 of the base holder 206. Each profile of a leg 218 may be substantially straight or may be curved and terminate in a foot portion 220. Each leg 218 may be substantially the same length. Each leg 218 may have the same shape or may have a different shape. A leg 218 may have smooth surfaces and smooth edges and either rounded or angular edges.

In one exemplary embodiment, each leg 218 may define a hole 225 preformed in the foot 220. A suture retention structure 229 may be formed in a top portion of each of the legs 218. The suture retention structure 229 may include a center cavity 221 formed in the top of each leg 218 defined between opposing sidewalls 235a, 235b. Each of the opposing sidewalls 235a, 235b may define an indented vertical channel 227a, 227b in an outer surface thereof. In the exemplary embodiment shown, the indented vertical channels 227 may be open at a bottom end (i.e., extend entirely to an edge of the respective leg 218) and have an arched form at a top end. Opposing apertures 233a, 233b may be formed beneath the arches at the top ends of the indented vertical channels 227a, 227b that open into the center cavity 221 immediately above and adjacent to a base wall of the center cavity 221. One of the opposing sidewalls 235a (in this exemplary embodiment, the left sidewall) may further define a saddle or trough 223 in a top surface thereof directly above, but spaced apart from, the indented vertical channel 227a on the left side of the leg 218. A cutting channel 231 may be formed as a narrow vertical channel within a center of the base of the center cavity 221 that extends radially inward to the core 216.

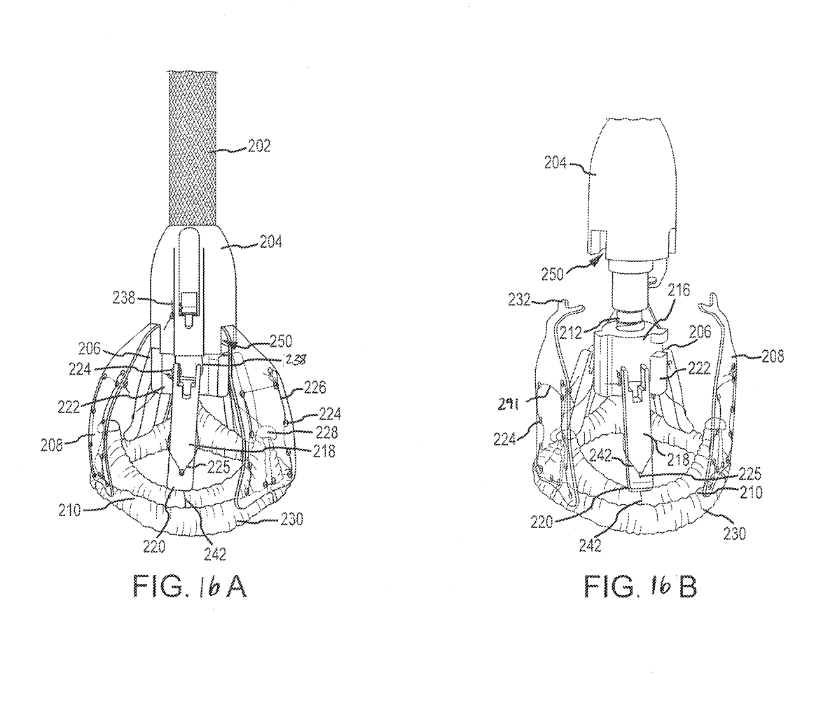

As shown in FIG. 16A and FIG. 16B, suture thread 242 may be used to attach each of the legs 218 of the base holder 206 to the valve base 210. By way of example, but not limitation, the suture thread 242 may be inserted through each of the opposing apertures 233a, 233b to extend across the center cavity 221. One end of the suture thread 242 may remain adjacent the left opposing aperture 233a. The suture thread 242 extends from the right opposing aperture 233b, down the right indented vertical channel 227b, behind the leg 218, around to the front of the leg 218, and through the hole 225 in the foot 220. The suture thread 242 passes through the foot 220 and further passes through and around a portion of the valve base 210, such as through the fabric cover of the valve base 210, the sewing cuff 230, or preformed holes 382 in the valve base 210 (see FIG. 21). The suture thread 242 next passes back through the hole 225 from the bottom to the top of the foot 220. The suture thread 242 wraps around the front to the right side of the leg 218, behind the leg 218, and up the left indented vertical channel 227a. The suture thread 242 then passes through the left opposing aperture 233a, upward within the center cavity 221, and around the trough 223 to meet with the first end of the suture thread 242. The free ends of the suture thread 242 are thereby tied in a knot on the outside of the left opposing sidewall 235a.

The threaded end 212 of the handle 202 may thread into an aperture 214 defined in the core 216 of a base holder 206. The aperture 214 may be positioned substantially in the center of the region defined by the circumference of the base holder 206. The inside of the aperture 214 may be threaded complimentary to the threaded end 212 of the handle 202. The aperture 214 may extend partially into or fully through the core 216 of the base holder 206. The threaded end 212 of the handle 202 may extend beyond the plane of the underside of the core 216 of the base holder 206, may be flush with the plane of the underside of the core 216 of the base holder 206, or may terminate above the plane of the underside of the core 216 of the base holder 206.

One or more, for example three, locking extensions 222 may be formed on the outer surface of the core 216 of the base holder 206 between adjacent legs 218. The locking extensions 222 may be substantially evenly spaced around the core 216 of the base holder 206. Each locking extension 222 may be substantially cuboidal in shape and may have smooth surfaces and rounded or angular edges. The thickness and/or width of the locking extension 222 may vary along the length of the locking extension 222. Each locking extension 222 may be of substantially the same length, which length may be approximately the length of the core 216 of the base holder 206. Each locking extension 222 may have the same shape or may have a different shape. For example, in the exemplary embodiment shown, one of the locking extensions 222 may be a monolithic, rectangular extension 243. In contrast, the other two locking extensions 222 may be formed in a first block 239 and a second block 237 that define a gap 241 between them. The shape of a locking extension 222 may aid in, for example, securing the base holder to other fixtures during packaging and/or storage.

A top surface of the first block 239 may be coplanar with a top surface of the core 216 and extend downward toward the gap 241 while the second block 237 may be substantially coplanar with a bottom surface of the core 216 and extend upward toward the gap 241 and terminate slightly above the tops of the legs 218.

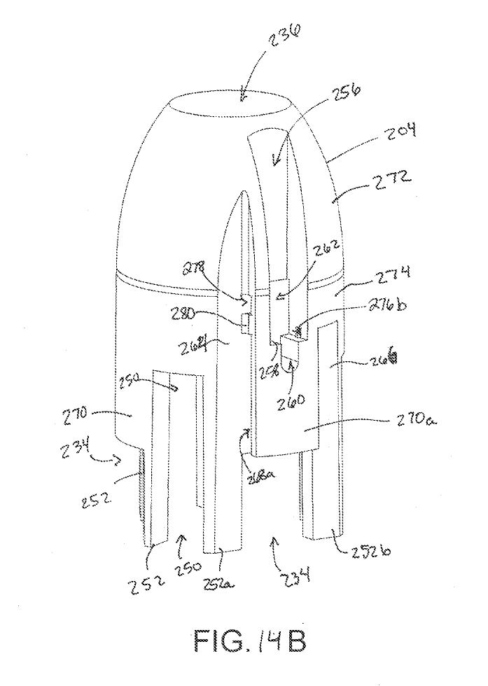

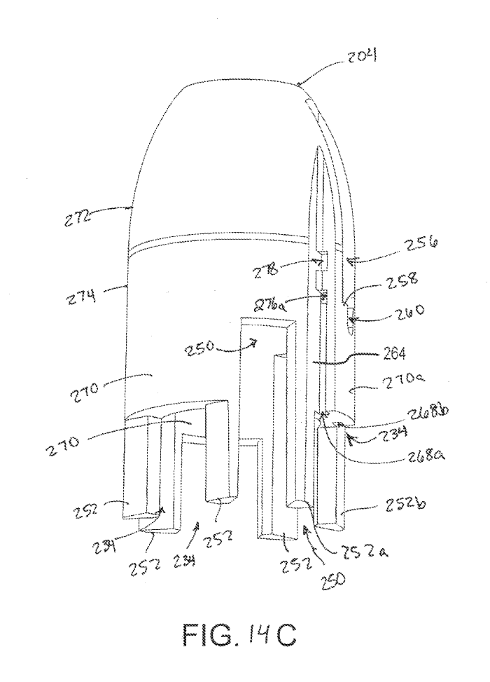

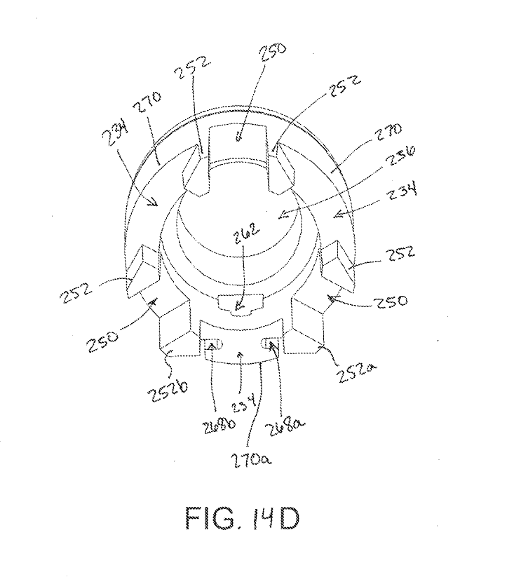

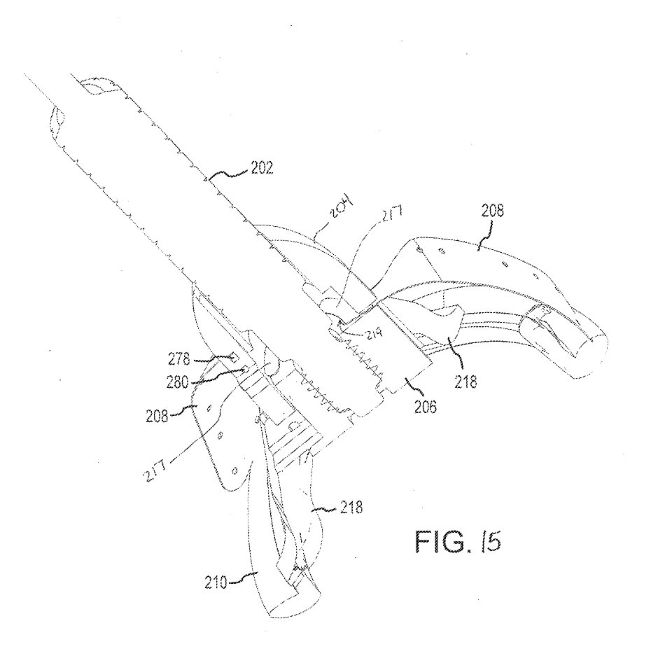

An exemplary embodiment of the shield cap 204 is shown in greater detail in FIGS. 14A-14D. The shield cap 204 may have an ellipsoidal top portion 272 that transitions into a cylindrical lower portion 274. The shield cap 204 may be formed of any biocompatible material known in the art including, but not limited to, plastic. The shield cap 204 may define a bore 236 through which the handle 202, including the threaded end 212, passes. The bore 236 may be positioned substantially in the longitudinal center of the shield cap 204. The configuration of the shield cap 204 and its position around the handle 202 ensures that the shield cap 204 does not fall off into the patient.

A number of guide legs 252, 252a, 252b may extend downward from a bottom of the cylindrical lower portion 274. The guide legs 252, 252a, 252b may define one or more, for example three, locking slots 250. The number of locking slots 250 may equal the number of retention tabs 232 and, correspondingly, the number of locking extensions 222. Each of the locking slots 250 may be substantially rectangular in shape, with a longer height than width. Each locking slot 250 may be the same or different shape.

In the embodiment shown, there are six guide legs 252, 252a, 252b that define three locking slots 250. The guide legs 252, 252a, 252b additionally define a number of leg slots 234, three in this embodiment, which are positioned between each adjacent pair of locking slots 250. The leg slots 234 in this embodiment are wider than the locking slots 250 while the locking slots 250 are taller than the leg slots 234. In this configuration, the wall forming the cylindrical lower portion 274 extends further downward above the leg slots 234 to form respective skirt sections 270, 270a. Pairs of the guide legs 252, 252a, 252b are configured to fit on either side of the opposing side walls 235a, 235b of a respective leg 218 of the valve base holder 206 and the leg slots 234 are configured to receive a top portion of a respective one of the legs 218 when the shield cap 204 is positioned on the valve base holder 206.

In the exemplary embodiment shown in FIGS. 14A-14D, one of the skirt sections 270a and the adjacent guide legs 252a, 252b are formed differently than the other corresponding areas of the shield cap 204. The outer faces 264, 266 of the guide legs 252a, 252b are formed or milled flat to expose lateral side edges of the skirt section 270a. The left outer face 264 extends along the shield cap 204 above the cylindrical lower portion and into the elliptical upper portion 272 while the right outer face 266 terminates at an upper edge within the lower cylindrical portion 274. Vertically oriented, recessed slots 268a, 268b may be formed within the lateral side edges of the skirt section 270a. The right recessed slot 268b may extend the length of the skirt section 270a to the top of the adjacent leg slot 234 while the left recessed slot 268a may extend the entire length of the lower cylindrical portion 274. A block 280 may be formed within the left recessed slot 268a to interrupt the left recessed slot below the termination at the top of the cylindrical portion 274, thereby forming a pocket 278 above the block 280.

A vertical chute 256 is formed within a sidewall of the shield cap 204 extending from a position in the lower cylindrical portion 274 slightly below the height of the right outer face 266 of the right guide leg 252b to end at an edge adjacent the top of the elliptical upper portion 272. The vertical chute 256 is substantially rectangular and tapers in depth from a base 258 in the lower cylindrical portion 274 to the terminal edge in the elliptical upper portion 272. A portion of the back wall of the vertical chute 256 may be open as a passage 262 to the bore 236 in a region generally limited to the lower cylindrical portion 274. A scissor slot 260 may be formed as a vertical channel in the base 258 of the vertical chute 256 extending downward into the skirt section 270a. A back of the scissor slot 260 may also be open to the bore 236 and form part of the passage 262.

A left attachment hole 276a may be formed within the left recessed slot 268a beneath the block 280 to extend into the vertical chute 256 above the base 258. A right attachment hole 276b may be formed within the right recessed slot 268b at the top edge to extend into the vertical chute 256 above the base directly opposing the left attachment hole 276a. The pocket 278 above the block 280 in the left recessed slot 268a may be formed as an aperture extending into the vertical chute 256 as well.

As shown in FIG. 15, the retention tabs 232 of the retraction shields 208 may be secured to the base holder 206 by way of the shield cap 204 which slides over the handle 202 to seat on top of the valve base holder 206. The retention tabs 232 of the retraction shields 208 may be secured between the underside of a top wall of the locking slots 250 and the top side of the locking extensions 222 of the base holder 206 which fit within the locking slots 250. The retention tabs 232 may be pinched between the underside of the locking slots 250 and the top side of the locking extensions 222. Alternatively, the retention tabs 232 may be positioned in a gap between the underside of the locking slots 250 and the top side of the locking extensions 222 such that the retention tabs 232 are secured without being touched and/or pinched by either or both of the locking slots 234 and the locking extensions 222. The retention tabs 232 may extend across the top side of the locking extensions 222 such that the ears 217 are positioned on the top surface of the core 216 of the base holder 206 and the curve of the top edge 219 follows the curve of the aperture 214 in the core 216.

The shield cap 204 may further be temporarily connected to the valve base holder 206 with suture thread 238 when the shield cap 204 is placed upon the base holder 206 as shown in FIG. 16A. The suture thread 238 may be inserted through each of the attachment holes 276a, 276b to extend across the vertical chute 256. One end of the suture thread 238 may remain adjacent the left attachment hole 276a. The suture thread 238 extends from the right attachment hole 276b, down the right recessed slot 268b, to one of the legs 218 directly below. The suture thread 238 then passes through the right opposing aperture 233b, across the center cavity 221, and out the left opposing aperture 233a. The suture thread 238 returns upward to the shield cap 204, along the left recessed slot 268a, and extends through the left attachment hole 276a into the vertical chute 256. The suture thread 238 then travels upward to the aperture of the pocket 278 and passes through the pocket 278 to exit above the block 280 to meet with the first end of the suture thread 238. The free ends of the suture thread 238 are thereby tied in a knot adjacent the block 280 between the pocket 278 and the left attachment hole 276a. While in the exemplary embodiment shown, only one of the legs 218 is attached to the shield cap 204 by the suture thread 238, the shield cap 204 could be configured to provide for attachment of each of the legs 218 to the shield cap 204.

By way of example, but not limitation, the valve introducer system and components of FIGS. 7-17 may be inserted into a patient undergoing aortic valve replacement surgery according to the following procedure. Note that a similar procedure may be followed for the implantation of an artificial pulmonary valve. After the natural aortic valve is resected, the valve base 210, its base holder 206, and the associated shields 208 are positioned above the surgical space by holding the handle 202. Sutures are then passed through the annular tissue and the sewing cuff 230 and suture apertures (hidden by the sewing cuff 230) in the valve base 210. Once all the sutures are attached between the annulus 118 and the valve base 210, the valve base 210 is pushed down into the patient's annulus 118 by pushing the base holder 206 with the handle 202. The sutures are then tied off by way of knots that are pushed down against the surface of the sewing cuff 230. The sutures may then be cut off, or left in place and organized by using the appropriate system for holding sutures, such as a Gabbay-Frater suture guide.

Once the valve base 210 is fully secured to the aortic root 116 by sutures through the sewing cuff 230, the retention tabs 232 of the retraction shields 208 may be released from the shield cap 204 by first cutting any securing suture thread 238 between the shield cap 204 and the legs 218 of the base holder 206. In order to cut the suture thread 238, a scissor may be inserted into the vertical chute 256 with the bottom blade of the scissor in the scissor slot 260 underneath the suture thread 238 for ease of cutting. Once the suture thread 238 is cut and removed, the shield cap 204 may be pulled upward to release the retention tabs 232 from between the locking slots 250 and locking extensions 222.

The base ends 207 of the retraction shields 208 are still secured to the valve base 210, for example by sutures 226. The retraction shields 208 are now free to be pushed or bent outward against tissue to provide retraction during further implantation of the valve base 210, connection of the valve leaflet set 110, and or reconnection of the aorta.

The effective length of a retraction shield 208 may be extended by a shield extender 134, for example of the type shown in FIG. 5A. Shield extenders 134 may be attached to the retraction shields 208 by sliding the head 136 of a shield extender 134 under the horizontal suture span 291 adjacent the top end of a retraction shield 208 such that the fingerhooks 138 capture the horizontal suture span 291. The shield extenders 134 may be attached to a Gabbay-Frater suture guide via a suture through a preformed hole 135 at the tail of a shield extender 134, or they may be clamped in a desired position with surgical clamps. Traction may be applied to the sutures that secure the shield extenders 134. This traction can be adjusted to provide the proper amount of exposure of the surgical site for additional suturing of the valve base 210 by way of soft tissue retraction, and also to effectively lift the annulus 118 of the aortic root 116 toward a more vertical orientation for the later installation of a leaflet set 110.

After the retention tabs 232 of the retraction shields 208 are released from between the shield cap 204 and the base holder 206, the valve base 210 can be released from the base holder 206, for example, by cutting any suture thread 242 holding the leg 218 of the base holder 206 to the valve base 210. In order to cut the suture thread 242, a scissor or a scalpel blade may be inserted into the center cavity 221 with the bottom blade of the scissor positioned in the cutting channel 231 underneath the suture thread 242 for ease of cutting. The base holder 206, shield cap 204, and handle 202 can then be removed from the surgical site. It should be noted that the base holder 206 can be removed at any desirable point during the procedure and need not be removed immediately after the retraction shields 208 are released.

The leaflet set 110 may be secured to hooks 228 extending from the valve base 210. Once the valve 102 is assembled in the patient, the sutures 226 can be cut to release both the retraction shields 208 and the attached shield extenders 134 to permit their removal from the surgical site.

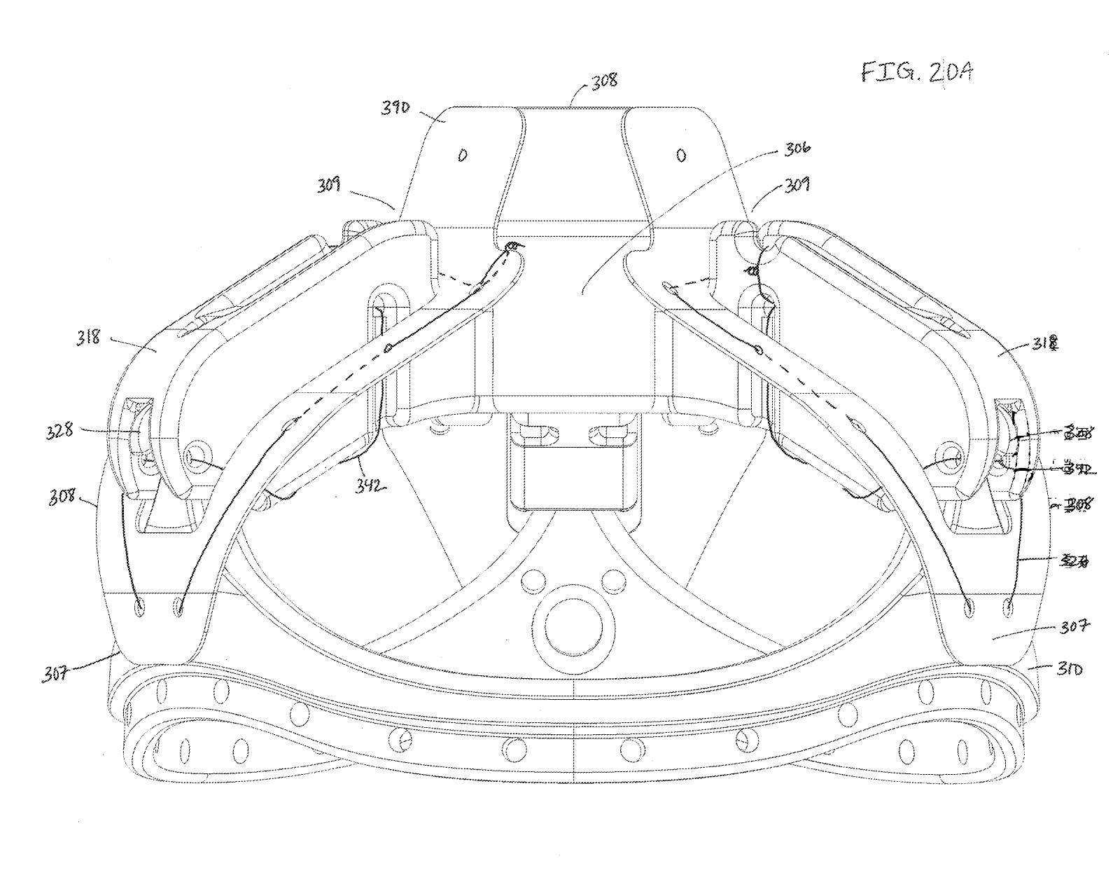

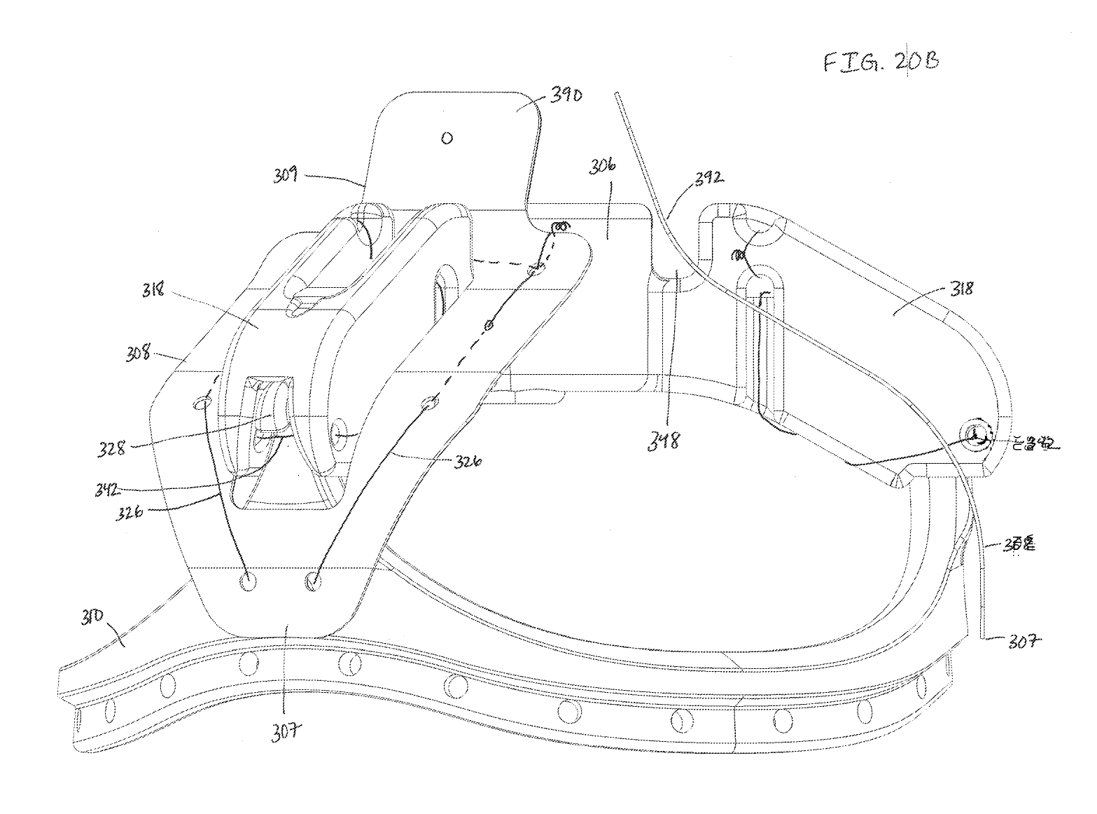

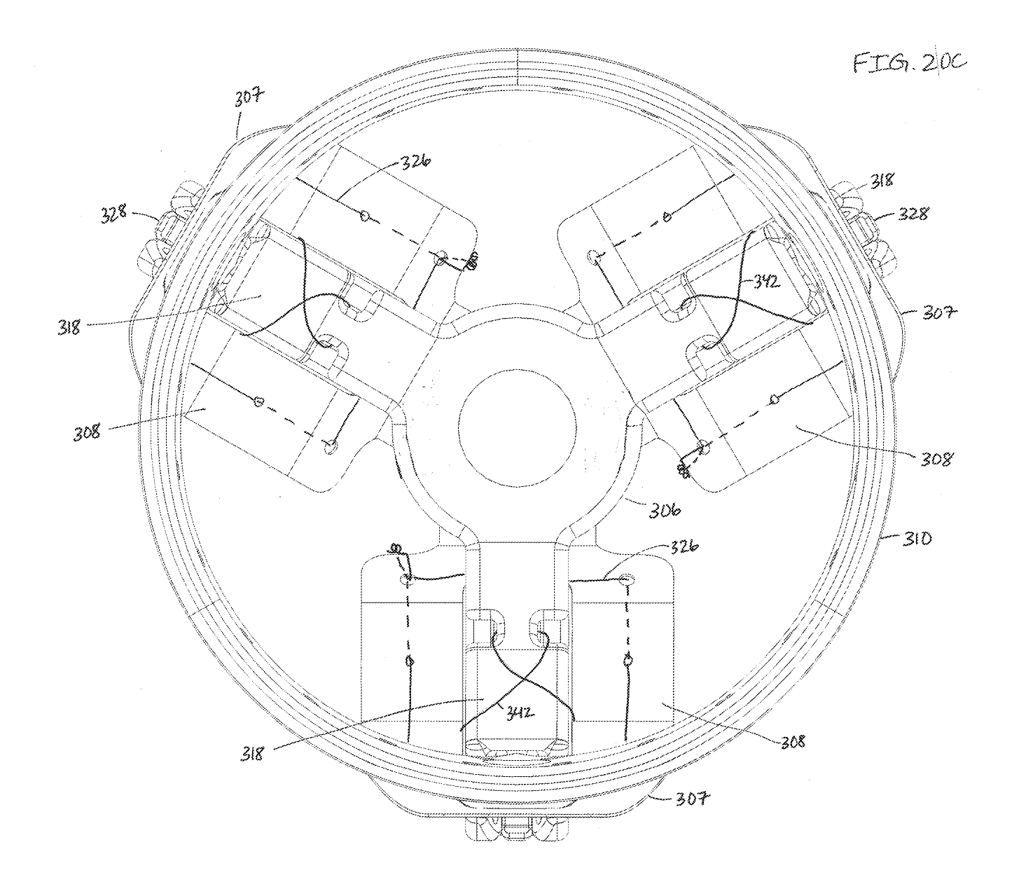

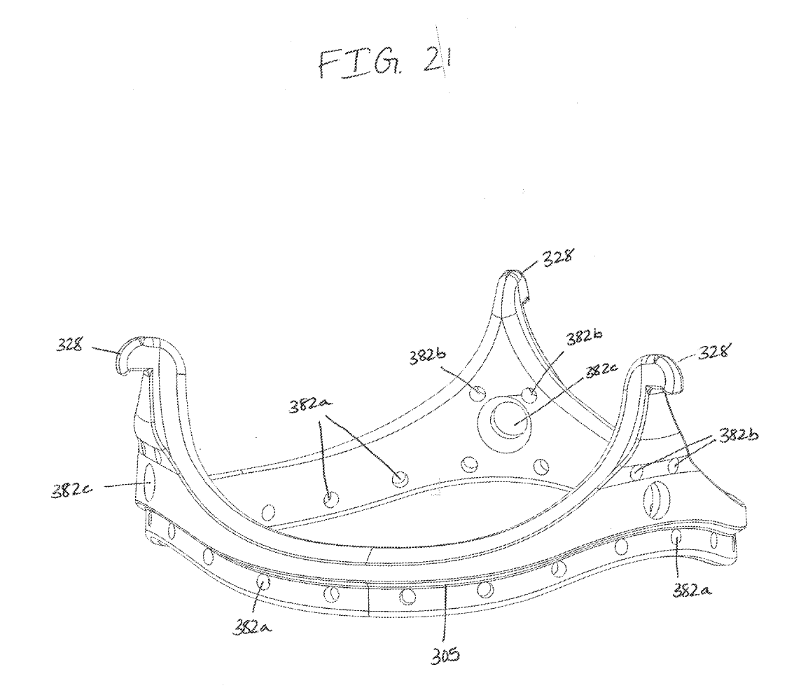

FIGS. 18-23 show a valve introducer system according to a third exemplary implementation that provides additional clearance and access to the surgical site as compared to the prior embodiments. With initial reference to FIGS. 18-22, a valve base 310 may be secured to a base holder 306 and the base holder 306 may be secured to an handle 302. One or more retraction shields 308 are secured at their base ends 307 to the valve base 310 and the free top ends 309 of the retraction shields 308 may be retained in a locking trough 348 of the base holder 306. One or more legs 318 of the base holder 306 fit over or around a respective one of one or more hooks 328 on the valve base 310 and secure the base holder 306 to the valve base 310.

The valve base 310 is comprised of one or more biocompatible materials. The biocompatible materials may be any biocompatible materials known in the art. By way of example but not limitation, the frame 305 (see FIG. 21) of the valve base 310 may be constructed of PEEK or medical grade stainless steel and it may be partially or completely covered with a woven polyester fabric (see FIG. 2).

As in the prior embodiments, the valve base 310, including the sewing cuff 330, may be substantially circular to fit in the annulus 118 (see FIG. 1). The valve base 310 may have smooth, curved edges. The underside of the valve base 310 may create a flat plane or it may undulate such that it falls above and/or below a plane, and so that it conforms to the shape of the aortic root 116. The top side of the valve base 310 rises in three places to form or secure a corresponding number of hooks 328. The hooks 328 are substantially evenly spaced around the circumference of the valve base 310. The hooks 328 face away from the interior of the space defined by the valve base 310 and may secure a leaflet set 110 (see FIG. 2) after the valve base 310 has been inserted into and attached to the aortic root 116 (see FIG. 1).

Any number of holes 382 may be preformed in the frame 305 of the valve base 310. By way of example, but not limitation, the preformed holes 382 may facilitate the attachment of the fabric cover of the valve base 310, facilitate attachment of the sewing cuff 330, and/or may aid in other assembly or installation of the valve 102. In the exemplary embodiment shown in FIG. 21, twenty four rim holes 382a are formed in the frame 305 of the valve base 310 adjacent and along the bottom surface of the valve base 310. The rim holes 382a may be substantially evenly spaced around the circumference of the valve base 310, or may be unevenly spaced. Two shield securing holes 382b are formed adjacent each other below and symmetric with a vertical center of each hook 328. One pin receiving hole 382c is formed directly below and centered between two shield securing holes 382b. All holes 382 may be substantially the same size, or the pin receiving holes 382c may be larger in diameter than the rim holes 382a and the shield securing holes 382b. The preformed holes 382 allow for passage of a needle and suture thread 326. The suture thread 326 may be inserted through some or all of the preformed holes 382.

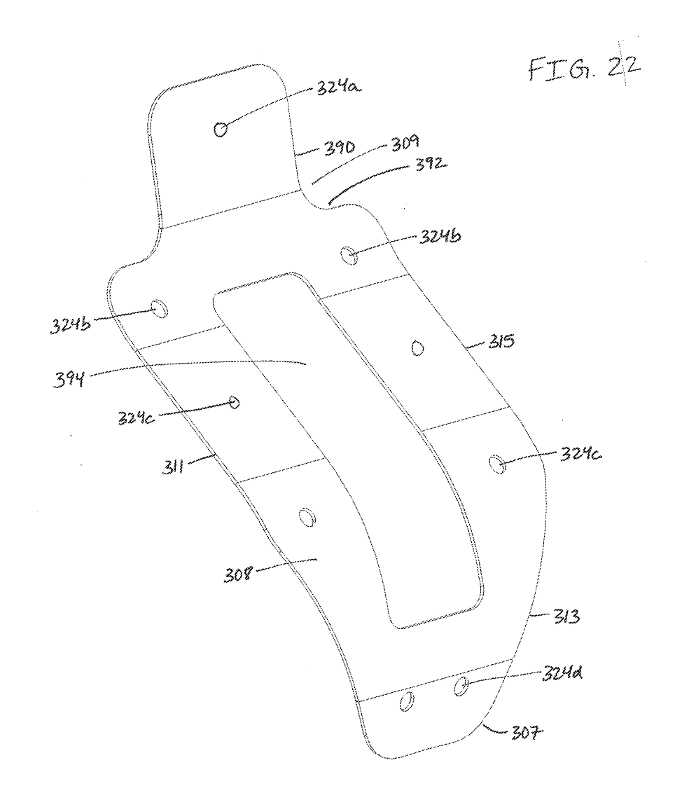

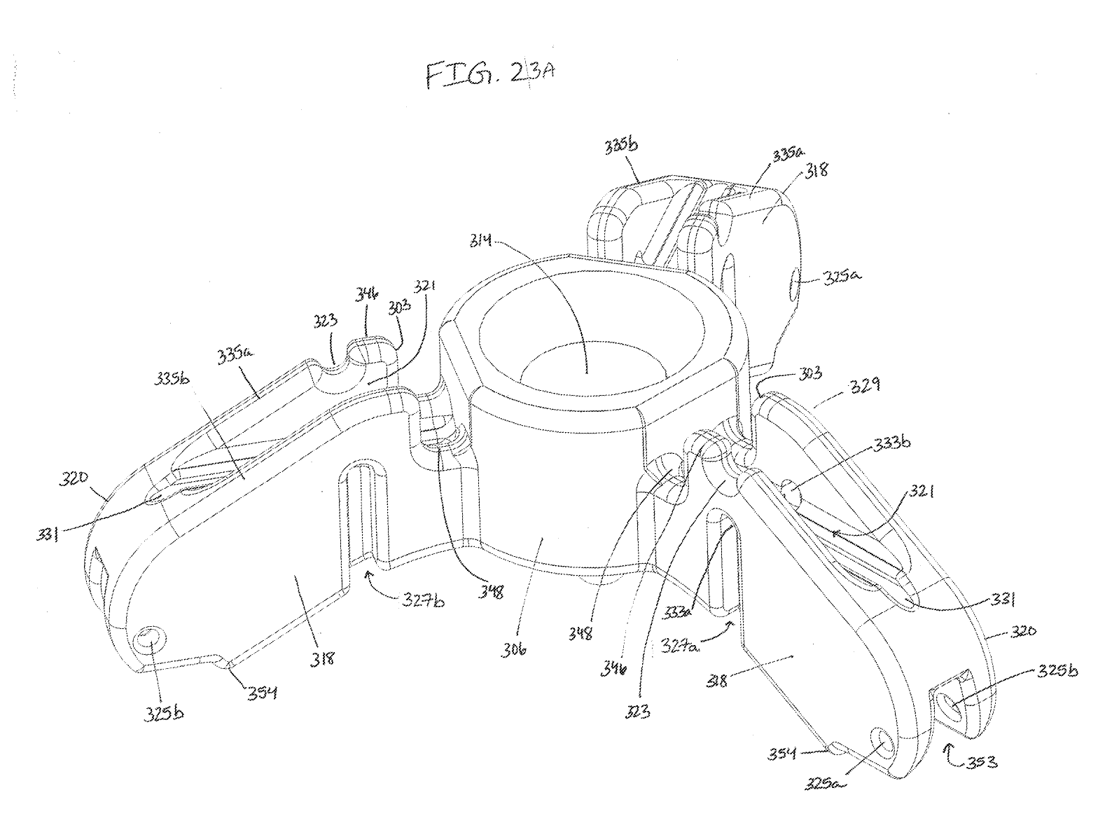

As shown in FIG. 22, each of the retraction shields 308 may have a substantially rectangular area 315 and a substantially triangular area 313 at the base end 307 of each shield 308. The width of the retraction shields 308 may vary over the length of the retraction shields 308. The retraction shields 308 are flexible to allow for attachment to the base holder 306 and later functionality as tissue retractors. For example, as shown in FIGS. 18-20, when attached to the base holder 306, each shield 308 may have a slightly S-shaped profile in which the top end 309 and base end 307 of each shield 308 curve in opposite directions compared to each other. The substantially rectangular area 315 may be substantially flat compared to the top end 309 and base end 307 of the retraction shield 308.

The sidewalls 311 of each shield 308 may turn inward toward the top end 309 of the retraction shield 308 to form a locking region 392. The top end 309 of each shield 308 may further be formed as a tab 390 substantially rectangular in shape. The retraction shields 308 may be formed of any sturdy and flexible biocompatible material known in the art. For example, the material may be plastic. The material may be transparent, translucent, or opaque. The retraction shields 308 may have smooth surfaces and smooth and/or rounded edges.

A leg aperture 394 may be formed in each shield 308 through which a leg 318 of the base holder 306 may pass. The leg aperture 394 may be positioned in the longitudinal center of shield 308. The leg aperture may be substantially rectangular in shape with rounded corners and a longer length than width. The width is wide enough to accommodate a leg 318 of the base holder 306.

Any number of holes 324 may be preformed in the retraction shields 308. By way of example, but not limitation, nine holes 324 may be preformed in the retraction shields 308 towards the outer edges of the retraction shields 308. In the exemplary embodiment shown in FIG. 22, two lower holes 324d are formed adjacent the base end 307 and adjacent each other symmetric with a longitudinal center of the retraction shield 308. Additionally, two sidewall holes 324c and one upper sidewall hole 324b may extend along and adjacent each of the sidewalls 311 in the rectangular area 315 of each shield 308. A tab hole 324a may be formed in the longitudinal center of the tab 390. The preformed holes 324 allow for passage of a needle and suture thread 326. The suture thread 326 may be inserted through some or all of the preformed holes 324.

With reference to FIGS. 18-20, the retraction shields 308 may be attached to the valve base 310. One or more sutures 326 may secure the base ends 307 of the retraction shields 308 to the valve base 310, for example, to the sewing cuff 330. As shown in the figures, a single suture thread 326 may be threaded through the holes 324 in each of the retraction shields 308 in a pattern around the perimeter of the retraction shields 308 and tied off adjacent an upper sidewall hole 324b adjacent the locking region 392. In the exemplary embodiment shown in FIG. 18, the suture thread 326 passes from a right upper sidewall hole 324b, under the locking region 392 of the retraction shield 308, and up through the left upper sidewall hole 324b. The suture thread 326 passes up and down through additional sidewall holes 324c on the left side of the retraction shield 308, passes through a lower hole 324d, and travels through the fabric cover of the valve base 310, the sewing cuff 330, and/or one or both of the shield securing holes 382b (see FIG. 21). The suture thread 326 then passes outward through the opposing lower hole 324d, up and down through the sidewall holes 324c on the right side of the retraction shield 308 to return to the right upper sidewall hole 324b and meet with the first end of the suture thread 326. The free ends of the suture thread 326 are thereby tied in a knot behind the right upper sidewall hole 324b.

The retraction shields 308 are positioned over the legs 318 of the valve base 310 with the legs 318 protruding through the leg aperture 394 of the retraction shields 308. Securing both the top end 309 and base end 307 of the retraction shields 308 permits the retraction shields 308 to be pulled against the base holder 306 during portions of an implantation procedure. Only the base ends 307 of the retraction shields 308 may be connected to the valve base 310 to permit the retraction shields 308 to be pushed or bent inward towards the base holder 306 and/or pushed or bent outward against tissue during implantation of the valve base 310, which may restrain tissue within the surgical site.

With reference to FIG. 18, the handle 302 may be formed of any sturdy biocompatible material. In an exemplary embodiment, the handle 302 may be made of stainless steel or titanium. The handle 302 may be constructed such that it is easy and comfortable for the human hand, including a gloved hand, to grip or hold. The handle 302 may be formed as an elongated shaft. The circumference of the handle 302 may vary over the length of the handle 302. The handle 302 may have a smooth surface or may be fully or partially cross-hatched, grooved, or ribbed. The handle 302 may be secured to the base holder 306 by a terminal threaded end 312. The handle 302 may be formed as a single piece or more than one assembled pieces.

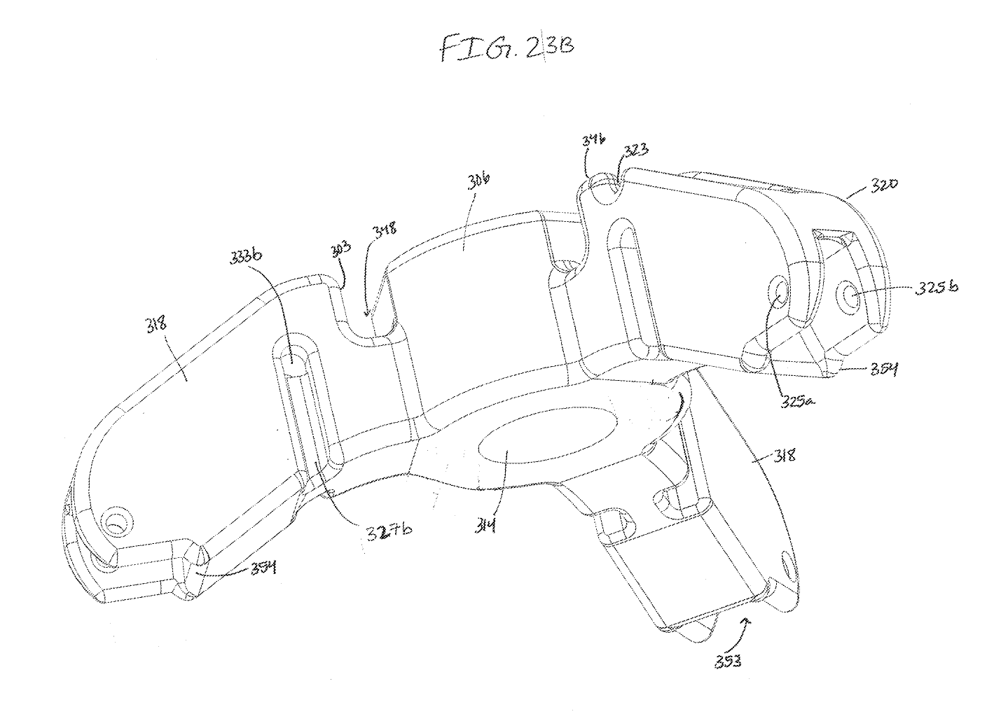

The base holder 306 is depicted in greater detail in FIG. 23A and FIG. 23B as a core 316 with a plurality of legs 318 extending therefrom. The base holder 306 may be formed of any biocompatible material known in the art including, but not limited to, plastic.

The core 316 is generally cylindrical but may have a flat face opposing a back wall 303 of each leg 318. A cylindrical threaded aperture 314 may be defined longitudinally in the center of core 316 of the base holder 306. The aperture 314 may extend partially into or fully through the core 316 of the base holder 306.

The plurality of legs 318, for example, three, may extend outward and slightly downward from the core 316 on the distal side of the locking trough 348. The legs 318 may be substantially evenly spaced around the core 316 of the base holder 306. Each profile of a leg 318 may be substantially straight but may curve downward slightly at its terminal end 320. Each leg 318 may be substantially the same length. Each leg 318 may have the same shape or may have a different shape. A leg 318 may have smooth surfaces and smooth edges and either rounded or angular edges. A locking trough 348 is formed by the outer surface of the core 316 and the back wall 303 of a leg 318. The locking trough 348 is at least deep enough to receive a locking region 392 of a shield 308.

In one exemplary embodiment, a suture retention structure 329 may be formed in a top portion of each of the legs 318. The suture retention structure 329 may include a center cavity 321 formed in the top of each leg 318 defined between opposing sidewalls 335a, 335b. Each of the opposing sidewalls 335a, 335b may define an indented vertical channel 327a, 327b in an outer surface thereof. In the exemplary embodiment shown, the indented vertical channels 327 may be open at a bottom end (i.e., extend entirely to an edge of the respective leg 318) and have an arched form at a top end. Opposing apertures 333a, 333b may be formed beneath the arches at the top ends of the indented vertical channels 327a, 327b that open into the center cavity 321 immediately above and adjacent to a base wall of the center cavity 321. One of the opposing sidewalls 335a (in this exemplary embodiment, the left sidewall) may further define a saddle or trough 323 in a top surface thereof directly above, but spaced apart from, the indented vertical channel 327a on the left side of the leg 318. The same sidewall 335a (in this exemplary embodiment, the left sidewall) may also define a ridge 346 between the back wall 303 of the leg 318 and the trough 323. A cutting channel 331 may be formed as a narrow vertical channel within a center of the base of the center cavity 321 that extends radially inward through the locking trough 348 and to the core 316.

The front face of a terminal end 320 of each leg 318 may define a hook cavity 353. The hook cavity 353 may be open at a bottom end (i.e., extend entirely to a bottom edge of the respective leg 318), be wide enough and deep enough to receive at least a potion of a hook 328, and have an arched interior and inward sloping sidewalls. Any number of holes 325a, 325b, for example two, may be preformed the sidewalls forming the hook cavity 353 in the terminal end 320 of each leg 318. The bottom edge of the leg 318 may be cut away to form a lip 354 on the underside of the terminal end 320 of the leg 318. The lip 354 may fit against the back of a hook 328 when the base holder 306 is secured to the valve base 310.

As shown in FIGS. 18-20, suture thread 342 may be used to attach each of the legs 318 of the base holder 306 to the valve base 310. In each case, the suture thread 342 may be inserted through each of the opposing apertures 333a, 333b to extend across the center cavity 321. One end of the suture thread 342 may remain adjacent the left opposing aperture 333a. The suture thread 342 extends from the right opposing aperture 333b, down the right indented vertical channel 327b, behind the leg 318, around to the left side of the leg 318, and through the left hole 325a in the terminal end 320 of the leg 318. The suture thread 342 passes through the hook cavity 353 and, if present, under a hook 328. The suture thread 342 then passes through the right hole 325b in the terminal end 320 of the leg 318, around to the right side of the leg 318, behind the leg 318, and up the left indented vertical channel 327a. The suture thread 342 then passes through the left opposing aperture 333a, upward within the center cavity 321, and around the trough 323 to meet with the first end of the suture thread 342. The free ends of the suture thread 342 are thereby tied in a knot on the outside of the left opposing sidewall 335a.

The retraction shields 308 may be secured to the base holder 306 by being seated over and around the legs 318 of the base holder 306. Each leg 318 fits through a leg aperture 394 of a shield 308. Each locking trough 348 of the base holder 306 receives a locking region 392 of the top end 309 of a shield 308. The flexible material of the retraction shields 308 may help the locking regions 392 fit within and remain secured in the locking troughs 348.

The threaded end 312 of the handle 302 may thread into the aperture 314 defined in the core 316 of the base holder 306. The inside of the aperture 314 may be threaded complimentary to the threaded end 312 of the handle 302. The threaded end 312 of the handle 302 may extend beyond the plane of the underside of the core 316 of the base holder 306, may be flush with the plane of the underside of the core 316 of the base holder 306, or may terminate above the plane of the underside of the core 316 of the base holder 306.

By way of example, but not limitation, the valve introducer system and components of FIGS. 18-23 may be inserted into a patient undergoing aortic valve replacement surgery according to the following procedure. Note that a similar procedure may be followed for the implantation of an artificial pulmonary valve. After the natural aortic valve is resected, the valve base 310, its base holder 306, and the associated shields 308 are positioned above the surgical space. Sutures are then passed through the annular tissue and the sewing cuff 330 and through the rim holes 382a in the valve base 310. Once all the sutures are attached between the annulus 118 and the valve base 310, the valve base 310 is pushed down into the patient's annulus 118 by pushing the base holder 306 with the handle 302. The sutures are then tied off by way of knots that are pushed down against the surface of the sewing cuff 330. The sutures may then be cut off, or left in place and organized by using the appropriate system for holding sutures, such as a Gabbay-Frater suture guide.

Once the valve base 310 is fully secured to the aortic root 116 by sutures through the sewing cuff 330, the retraction shields 308 may be released from the base holder 306 by pulling upward on each tab 390 to lift the top end 309 of each shield 308 out of the locking trough 348 between the legs 318 and the core 316 of the base holder 306.

The base ends 307 of the retraction shields 308 are still secured to the valve base 310, for example by sutures 326. The retraction shields 308 are now free to be pushed or bent outward against tissue during further implantation of the valve base 310 and connection of the valve leaflet set 110.

The effective length of a shield 308 may be extended by a shield extender 134, for example of the type shown in FIG. 5A. Shield extenders 134 may be attached to the retraction shields 308 by sliding the head 136 of a shield extender 134 under the horizontal suture span 391 passing across a top surface of the locking region 392 each shield 308 between upper sidewall holes 324b such that the fingerhooks 138 capture the horizontal suture span 391. Alternatively, each shield extender 134 may be secured to a leg aperture 394 of a respective shield 308, such as via a hook-shaped end of the shield extender 134.

The shield extenders 134 may be attached to a Gabbay-Frater suture guide via a suture through a preformed hole 135 at the tail of a shield extender 134, or by clamping them with surgical clamps. Traction may be applied to the sutures that secure the shield extenders 134. This traction can be adjusted to provide the proper amount of exposure of the surgical site for additional suturing of the sewing cuff 330 and frame 305 of the valve base 310 by way of soft tissue retraction, and also to effectively lift the annulus 118 of the aortic root 116 toward a more vertical orientation for the later installation of a leaflet set 110.

After the retraction shields 308 are released from the legs 318 of the base holder 306, the valve base 310 can be released from the base holder 306, for example, by cutting any suture thread 342 holding the legs 318 of the base holder 306 to the valve base 310. In order to cut the suture thread 342, a scissor or scalpel blade may be inserted into the center cavity 321 with the bottom blade of the scissor positioned in the cutting channel 331 underneath the suture thread 342 for ease of cutting. The base holder 306 and handle 302 can then be removed from the surgical site. It should be noted that the base holder 306 can be removed at any desirable point during the procedure and need not be removed immediately after the retraction shields 308 are released.

The leaflet set 110 may be secured to hooks 328 extending from the valve base 310. Once the valve 102 is assembled in the patient, the sutures 326 can be cut to release both the retraction shields 308 and the attached shield extenders 134 to permit their removal from the surgical site.

The system thus described may be understood to confer several benefits. First, the shields or legs may provide a covering for the hooks of the valve base while the valve base is being implanted. The shields may further provide an easy path for knots to follow as they are pushed down against the sewing cuff. Second, the combination of shields and shield extenders may provide traction and soft tissue retraction that allows easier access to the valve base while the leaflet set is being inserted. Third, the shields may be used as a valve base lifter that can orient the valve base while in the patient's heart for easier access during leaflet insertion or during examination of the implanted valve. Fourth, the tissue retraction shields may aid in minimizing the invasiveness of the procedure by reducing the size of the surgical incision to 7 cm or less because the available tissue retraction provided by the shields greatly opens up the sight into the surgical field.

All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present invention, and do not create limitations, particularly as to the position, orientation, or use of the invention. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. The exemplary drawings are for purposes of illustration only and the dimensions, positions, order and relative sizes reflected in the drawings attached hereto may vary.

The above specification, examples and data provide a complete description of the structure and use of exemplary embodiments of the invention as defined in the claims. Although various embodiments of the claimed invention have been described above with a certain degree of particularity, or with reference to one or more individual embodiments, those skilled in the art could make numerous alterations to the disclosed embodiments without departing from the spirit or scope of the claimed invention. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

What is claimed is:

1. A bioprosthetic cardiac valve base configured for attachment to a separate leaflet set, the valve base comprising
a substantially annular valve seat provided with a biocompatible fabric coating;
a plurality of hooks extending proximally from the valve seat and configured for attachment with a valve leaflet set; and
a plurality of retraction shields for restraining tissue within a surgical site, each retraction shield positioned adjacent to and extending over a respective one of the plurality of hooks, wherein a base end of each of the retraction shields is realeasably secured to the valve seat.

2. The valve base of claim 1, wherein each of the retraction shields terminates at a top end to form a retention tab configured for capture within an introducer device.

3. The valve base of claim 1, wherein the retraction shields are formed of a biocompatible, flexible, transparent material.

4. The valve base of claim 1, wherein a plurality of holes is preformed in each of the retraction shields.

5. The valve base of claim 4, wherein a base end of each of the retraction shields is secured to the valve base with sutures placed through one or more of the plurality of holes.

6. The valve base of claim 1 further comprising a shield extender secured to one of the retraction shields.

7. The valve base of claim 4 further comprising
a suture thread threaded through at least two of the plurality of holes on one of the retraction shields to form a span of suture thread on an outer surface of the one of the shields; and
a shield extender secured to the span of suture thread.

8. The valve base of claim 7, wherein the shield extender has an arrow-shaped head formed with two tail fingerhooks that capture the span of suture thread on the outer surface of the one of the retraction shields.

9. The valve base system of claim 8, wherein the shield extenders are configured to secure to a device for providing tissue retraction during a surgical procedure.

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. A prosthetic heart valve implantation system comprising
a valve base configured for in vivo implantation and to hold a valve leaflet set; and
a tissue retraction shield removably attached to the valve base.

42. A method of implanting a prosthetic cardic valve comprising
suturing a valve base to an implantation site on a heart; and
retracting tissue surrounding the implantation site by placing traction on one or more retraction shields removably attached to the valve base.

43. The method of claim 42 further comprising attaching retraction extenders to the retraction shields.

44. The method of claim 42 further comprising attaching the retraction shields to the valve base.

45. The method of claim 42 further comprising detaching the retraction shields from the valve base.

46. The method of claim 42 further comprising attaching a removable leaflet set to the valve base.

47. The method of claim 42 further comprising making an incision of 7 cm or less for exposure of a surgical field.

48. The system of claim 41 further comprising a shield extender secured to the retraction shield.

49. The system of claim 48, wherein the shield extender is configured to secure to a device for providing tissue retraction during a surgical procedure.

50. The system of claim 48 further comprising an elongated handle;
a valve base holder attached to a distal end of the handle and realeasably secured to the valve base;
a shield retainer moveably positioned on the handle above the valve base holder; wherein
a top end of the retraction shield is releasably secured to the shield retainer.

51. The system of claim 50, wherein
the valve base holder further comprises a leg extending therefrom; and
a distal end of the leg is releasably secured to the valve base.
